(19) United States
(12) Patent Application Publication
(10) Pub. No.: US 2021/0350909 A1
(43) Pub. Date: Nov. 11, 2021

(54) System, Method and/or Computer Readable Medium For Optimizing Patient Rehabilitation

(71) Applicant: Bionik, Inc., Watertown, MA (US)

(72) Inventors: Abhijit Saxena, Toronto (CA); Malcolm Bock, Medfield, MA (US); Florian Gmeiner, Toronto (CA); Eric Dusseux, Paris (FR); Michal Prywata, Toronto (CA); Sherri Wallis, Springtown, TX (US); Loren Wass, Kennebunk, ME (US)

(73) Assignee: Bionik, Inc., Watertown, MA (US)

(21) Appl. No.: 17/091,041

(22) Filed: Nov. 6, 2020

Related U.S. Application Data
(60) Provisional application No. 63/023,199, filed on May 11, 2020.

(51) Int. Cl. G16H 40/20; G16H 40/67; G16H 15/00; G16H 10/60; G16H 50/70; G16H 50/20; G06Q 40/08; G06Q 10/10; G06Q 50/04

(52) U.S. Cl. CPC G16H 40/20; G16H 40/67; G16H 15/00; G16H 10/60; G06Q 50/04; G16H 50/20; G06Q 40/08; G06Q 10/10; G16H 50/70

(57) ABSTRACT

Disclosed is a system, method and/or computer readable medium for use with one or more rehabilitation devices to optimize patient rehabilitation by engaging multiple stakeholders in the patient rehabilitation process to generate one or more positive feedback loops.

### FIELD OF THE INVENTION

The present invention relates generally to a system for use with a rehabilitation device. In particular, the present invention relates to a system, method and/or computer readable medium for use with a rehabilitation device to optimize patient rehabilitation.

### BACKGROUND OF THE INVENTION

In the prior art, different solutions have been developed for rehabilitating patients who may have experienced motor impairments including stroke, cerebral palsy, spinal cord injury, multiple sclerosis, Parkinson's disease, hemiplegic shoulder pain and/or muscle spasticity. The prior art attempts, however, have been limited to manual (i.e., low usage and/or adoption of technology) rehabilitation and little to no visibility of the patient's rehabilitation progress. These limitations may have resulted in sub-optimal or delayed rehabilitation progress.

Accordingly, it is a problem in the art to develop technology-based rehabilitation solutions capable of providing stakeholders with visibility (over a predetermined amount of time, including in real-time) on the patient's rehabilitation progress.

As a result, there may be a need for, or it may be desirable to provide a holistic approach in the rehabilitation facility and/or cooperating environment that overcomes one or more of the limitations associated with the prior art.

### SUMMARY OF THE INVENTION

The present disclosure provides a system, method and/or computer readable medium for optimizing patient rehabilitation.

According to an aspect of one preferred embodiment of the invention, there is disclosed a system for optimizing one or more clinical outcomes for a rehabilitation patient by a stakeholder or authorized user. The system includes a rehabilitation subsystem having: (i) a device adapted to generate device data associated with the rehabilitation patient; and (ii) one or more rehabilitation processors operative to electronically receive and transmit the device data. In addition, the system includes an administrator subsystem having: (i) one or more administrator processors operative to electronically receive the device data from the one or more rehabilitation processors and to automatically apply an administrator algorithm to the device data to generate one or more reports associated with the rehabilitation patient; and (ii) a database for electronically storing the device data and the one or more reports associated with the rehabilitation patient. The system also includes an accessing entity subsystem comprising an accessing entity processor operative to execute a stakeholder facing application for reviewing at least a portion of the device data and/or the one or more reports associated with the rehabilitation patient from the database by the stakeholder. Thus, according to the invention, the system is operative to facilitate the optimization of the one or more clinical outcomes for the patient based on the review of the at least a portion of the device data and/or the one or more reports associated with the rehabilitation patient by the stakeholder.

According to an aspect of one preferred embodiment of the invention, the system, method and/or computer readable medium optimizes patient rehabilitation by improving device utilization, providing patient outcomes information, facilitating payment of insurance claims, providing clinical guidance and training, facilitating continuous development and improvement, providing clear documentation of patient progress, generating easy to read reporting, generating visual outcomes for each rehabilitation device, determining average session duration, determining average repetitions per session, determining the number of clinicians using a device, determining the total number of sessions for a patient and/or device, determining the total number of patients using the devices, and/or facilitating improved therapy outcomes for the patient.

According to an aspect of one preferred embodiment of the invention, the system, method and computer readable medium encourages the adoption of technology in-patient rehabilitation by enhancing transparency with respect to patient progress (e.g., patient use of the device, corporate management determining how much the device is being used, etc.) and/or providing dashboards adapted to present data and/or reports dynamically (i.e., in real-time, updatable and/or customizable) to the various stakeholders.

According to an aspect of one preferred embodiment of the invention, the system, method and computer readable medium is adapted to optimize patient rehabilitation by facilitating a continuum of care for the patient. In particular, data associated with the patient's rehabilitation progress is visible to authorized stakeholders and moves with the patient as they transfer between healthcare facilities and home.

According to a preferred embodiment, there is provided a system for optimizing one or more clinical outcomes for a rehabilitation patient by a stakeholder. The system includes a rehabilitation device for generating device data associated with the rehabilitation patient, the device including one or more rehabilitation processors operative to electronically receive and/or transmit the device data. The system also includes one or more administrator processors operative to: (i) electronically receive the device data from the one or more rehabilitation processors; and (ii) automatically apply an administrator algorithm to the device data to generate one or more reports associated with the rehabilitation patient. One or more databases are provided for electronically storing the device data and the one or more reports associated with the rehabilitation patient. Also included in the system is an accessing entity processor operative to execute a stakeholder facing application for reviewing at least a portion of the device data and/or the one or more reports associated with the rehabilitation patient from the one or more databases by the stakeholder. The system is operative to facilitate the optimization of the one or more clinical outcomes for the patient based on the review of the at least a portion of the device data and/or the one or more reports associated with the rehabilitation patient by the stakeholder.

According to an aspect of a preferred embodiment of the invention, the system further includes a secure connection module to facilitate the secure communication of data between the one or more rehabilitation processors and the one or more administrator processors.

According to an aspect of a preferred embodiment of the invention, the system further includes a plurality of rehabilitation devices.

According to an aspect of a preferred embodiment of the invention, the system further includes two or more rehabilitation facilities.

According to an aspect of a preferred embodiment of the invention, the system further includes update data and the one or more administrator processors are operative to transmit the update data to the one or more rehabilitation processors.

According to an aspect of a preferred embodiment of the invention, the administrator processor is further adapted to transmit the device data received from the rehabilitation processor to a second rehabilitation processor.

According to an aspect of a preferred embodiment of the invention, the reports include a management report, a robot utilization report, an outcome report, an individual patient evaluation and therapy log report, an individual patient therapy report, and/or an individual patient evaluation report.

According to an aspect of a preferred embodiment of the invention, the rehabilitation patient includes patients with nontraumatic epidural haemorrhage, nontraumatic subdural haemorrhage, multiple sclerosis, intracerebral haemorrhage, parkinsonism, motor neuron disease, and/or spastic cerebral palsy.

According to an aspect of a preferred embodiment of the invention, the stakeholder includes a patient, a patient family member, a patient friend, a clinician, a rehabilitation manager, a hospital manager, an insurer and/or a device manufacturer.

According to an aspect of a preferred embodiment of the invention, the optimization of the one or more clinical outcomes for the patient includes an increase in a Rose plot and/or an increase in FMA-UE score associated with the rehabilitation patient.

In accordance with a preferred embodiment, there is provided a method for optimizing one or more clinical outcomes for a rehabilitation patient by a stakeholder. The method includes the steps of: (a) operating a rehabilitation device to generate device data associated with the rehabilitation patient; (b) operating one or more rehabilitation processors to collect the device data associated with the rehabilitation patient and electronically transmit the data; (c) operating one or more administrator processors to electronically receive the device data from the one or more rehabilitation processors to: (i) automatically apply an administrator algorithm to the device data to generate one or more reports associated with the rehabilitation patient; (d) electronically storing the device data and the one or more reports associated with the rehabilitation patient; and (e) operating an accessing entity processor to execute a stakeholder facing application for reviewing at least a portion of the device data and/or the one or more reports associated with the rehabilitation patient from the one or more databases by the stakeholder. One or more clinical outcomes for the patient are optimized based on the review of the at least a portion of the device data and/or the one or more reports associated with the rehabilitation patient by the stakeholder.

According to an aspect of a preferred embodiment of the invention, the method further includes a step of operating a secure connection module to facilitate the secure communication of data between the one or more rehabilitation processors and the one or more administrator processors.

According to an aspect of a preferred embodiment of the invention, the method further includes a step of transmitting update data from the one or more administrator processors to the one or more rehabilitation processors.

According to an aspect of a preferred embodiment of the invention, the method further includes a step of transmitting the device data received form the rehabilitation processor to a second rehabilitation processor.

According to an aspect of a preferred embodiment of the invention, the reports of the method include a management report, a robot utilization report, an outcome report, an individual patient evaluation and therapy log report, an individual patient therapy report, and/or an individual patient evaluation report.

According to an aspect of a preferred embodiment of the invention, the rehabilitation patients of the method include patients with nontraumatic epidural haemorrhage, nontraumatic subdural haemorrhage, multiple sclerosis, intracerebral haemorrhage, parkinsonism, motor neuron disease, and/or spastic cerebral palsy.

According to an aspect of a preferred embodiment of the invention, the stakeholders of the method include a patient, a patient family member, a patient friend, a clinician, a rehabilitation manager, a hospital manager, an insurer and/or a device manufacturer.

According to an aspect of a preferred embodiment of the invention, the optimization of the one or more clinical outcomes for the patient by the method includes an increase in a Rose plot and/or an increase in FMA-UE score associated with the rehabilitation patient.

In accordance with a preferred embodiment of the invention, there is provided a non-transient computer readable medium on which is physically stored executable instructions for use with optimizing one or more clinical outcomes for a rehabilitation patient by a stakeholder, wherein the executable instructions include processor instructions for one or more rehabilitation processors, one or more administrator processors and/or an accessing entity processor to automatically: (a) generate device data associated with the rehabilitation patient; (b) apply an administrator algorithm to the device data to generate one or more reports associated with the rehabilitation patient; (c) electronically store the device data and the one or more reports associated with the rehabilitation patient in one or more databases; and (d) execute a stakeholder facing application for reviewing at least a portion of the device data and/or the one or more reports associated with the rehabilitation patient from the one or more databases by the stakeholder. The review of the at least a portion of the device data and/or the one or more reports associated with the rehabilitation patient by the stakeholder are for use in optimizing the one or more clinical outcomes for the patient.

Other advantages, features and characteristics of the present invention, as well as methods of operation and functions of the related elements of the system, method and/or computer readable medium for optimizing patient rehabilitation, and the combination of steps, parts and economies of manufacture, will become more apparent upon consideration of the following detailed description and the appended claims with reference to the accompanying drawings, the latter of which are briefly described herein below.

The novel features which are believed to be characteristic of the system, method and/or computer readable medium according to the present invention, as to their structure, organization, use, and method of operation, together with further objectives and advantages thereof, may be better understood from the following drawings in which presently preferred embodiments of the invention may now be illustrated by way of example. It is expressly understood, however, that the drawings are for the purpose of illustration and description only and are not intended as a definition of the limits of the invention

### BRIEF DESCRIPTION OF THE SEVERAL VIEWS OF THE DRAWINGS

FIG. 1 is a perspective view of the InMotion ARM/HAND™ robot employing the hand robot assembly in accordance with an embodiment of the present invention.

FIGS. 2A-C are collectively a schematic diagram of a system for optimizing patient rehabilitation in accordance with a preferred embodiment.

FIG. 3 is a schematic diagram of stakeholders associated with the system in accordance with a preferred embodiment.

FIG. 4 is a schematic diagram of one-way data flow for the system in accordance with a preferred embodiment.

FIG. 5 is a schematic diagram of two-way data flow for the system in accordance with a preferred embodiment.

FIG. 6 is a schematic diagram of data flow between facilities for the system in accordance with a preferred embodiment.

FIG. 7 is a collective overview report (monthly) for multiple sites generated by the system in accordance with a preferred embodiment.

FIG. 8 is a collective overview report (monthly) for a specific site generated by the system in accordance with a preferred embodiment.

FIG. 9 is a collective overview report (two-week period) for a specific site generated by the system in accordance with a preferred embodiment.

FIG. 10 is a tabular data report generated by the system in accordance with a preferred embodiment.

FIG. 11 is a device connection status report generated by the system in accordance with a preferred embodiment.

FIG. 12 is a flow diagram of a method of optimizing patient rehabilitation in accordance with a preferred embodiment.

FIG. 13 is a flow diagram of a reimbursement generation model in accordance with a preferred embodiment.

FIG. 14 is an example of a robot utilization report in accordance with a preferred embodiment.

FIG. 15 is an example of an outcome report in accordance with a preferred embodiment.

FIG. 16 is an example of an individual patient evaluation and therapy log in accordance with a preferred embodiment.

FIG. 17 is an example of an individual patient therapy report in accordance with a preferred embodiment.

FIGS. 18A-E are examples of a circle assessment, a point-to-point assessment, a stabilization assessment, a resistance assessment, and a progress summary result, respectively, in accordance with a preferred embodiment.

### DETAILED DESCRIPTION OF THE PREFERRED EMBODIMENTS

The description that follows, and the embodiments described therein, may be provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and, in some instances, proportions may have been exaggerated in order to more clearly depict certain embodiments and features of the invention.

The present disclosure may be now described in terms of an exemplary system in which the present disclosure, in various embodiments, would be implemented. This may be for convenience only and may not be intended to limit the application of the present disclosure. It may be apparent to one skilled in the relevant art(s) how to implement the present disclosure in alternative embodiments.

Certain novel features which are believed to be characteristic of a system for a rehabilitation apparatus which are novel in conjunction with the cooperating environment, according to the present invention, as to their organization, use, and/or method of operation, together with further objectives and/or advantages thereof, may be better understood from the accompanying disclosure in which presently preferred embodiments of the invention are disclosed by way of example. It is expressly understood, however, that the accompanying disclosure is for the purpose of illustration and/or description only and is not intended as a definition of the limits of the invention.

InMotion Rehabilitation Device

The described embodiments provide an apparatus for providing motor therapy that combines reaching with grip and release movements. The InMotion ARM/HAND™ apparatus is provided by BIONIK LABORATORIES CORP. (Toronto, ON, Canada). The InMotionHand™ and InMotion Arm™ robots, as described in U.S. patent application Ser. No. 16/719,821 (incorporated herein by reference), quietly monitor the patient's movements during therapy and may gently assist as needed to help the patient complete various motor therapy activities. InMotion robots may be used by patients experiencing a wide range of motor impairments including stroke, cerebral palsy, spinal cord injury, multiple sclerosis, Parkinson's disease, hemiplegic shoulder pain and/or muscle spasticity. Although the current embodiment is described in relation to the InMotion ARM/HAND™ apparatus, persons having ordinary skill in the art will understand that any other rehabilitation devices and/or robots may also be used with the invention to provide the same advantages such as, for example, the InMotion portable rehabilitation apparatus, as described in U.S. patent application Ser. No. 16/997,145 (incorporated herein by reference).

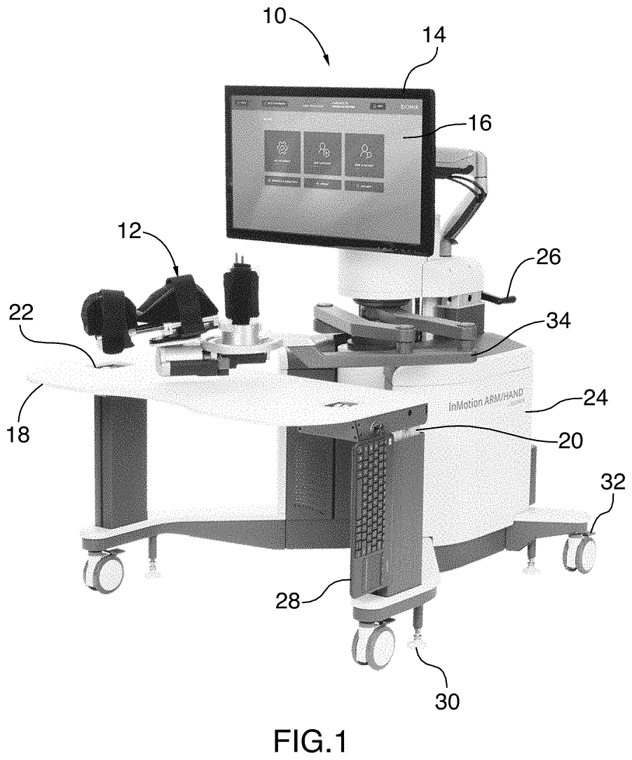

As shown in FIG. 1, the InMotion ARM/HAND™ apparatus 10 is an integrated patient workstation that, among others, includes: a hand and arm support assembly 12; a monitor 14; a monitor screen 16 for use by the patient; a table 18; a table height adjustment 20 to accommodate patients of different sizes; a stop button 22 to terminate the therapy session; a control box 24 containing the processor (i.e., the “rehabilitation processor 24”), memory and/or database; a robot height adjustment 26 to accommodate patients of different sizes; an input device 28 (e.g., keyboard); leveling feet 30 (four total); wheel locks 32 (4 total); and support structure 34. In addition, the InMotion apparatus 10 provides easy cleaning of smooth surfaces, hidden hand mounting screws, reduced pinch points and/or hidden cables when possible within the support structure.

System

The system preferably optimizes clinical engagement, patient engagement, patient outcomes and device utilization within the rehabilitation industry.

Embodiments of the system described herein may be implemented in hardware or software, or a combination of both. These embodiments may be implemented in computer programs or algorithms executing on programmable computers, each computer including at least one processor, a data storage system (including volatile memory or non-volatile memory or other data storage elements or a combination thereof), and at least one communication interface.

Each program may be implemented in a high-level procedural or object-oriented programming or scripting language, or both. Alternatively, the programs or algorithms may be implemented in assembly or machine language, if desired. The language may be a compiled or interpreted language. Each such computer program may be stored on a non-transitory computer-readable storage medium (e.g., read-only memory, magnetic disk, optical disc). The storage medium so configured causes a computer to operate in a specific and predefined manner to perform the functions described herein.

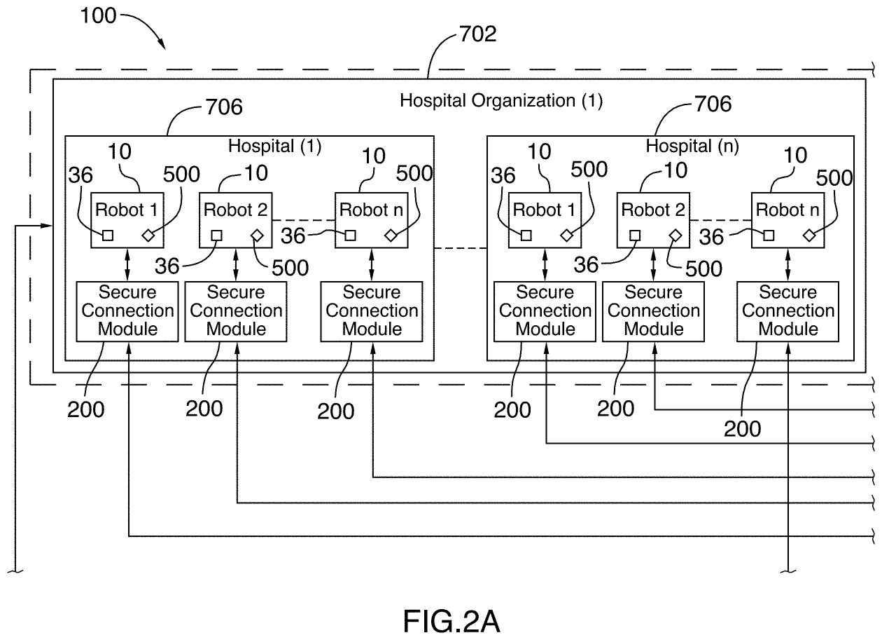

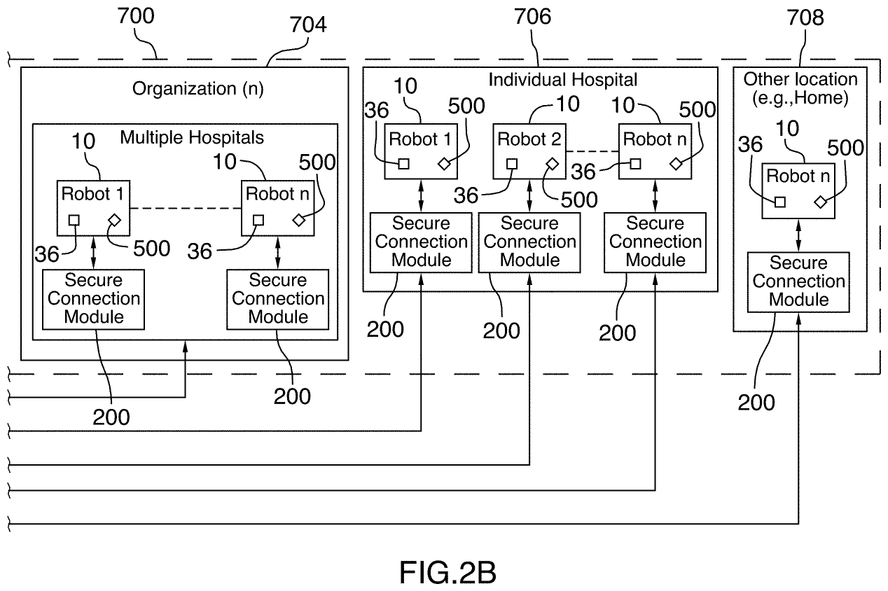

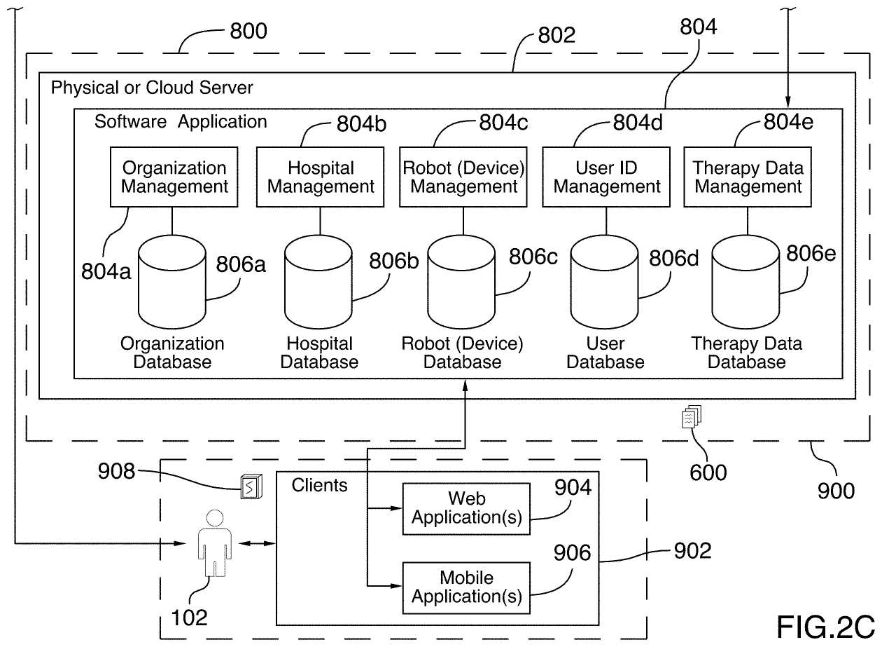

Referring now to FIGS. 2A-C, there is shown a schematic of a system 100 for one or more rehabilitation devices 10, such as the InMotion ARM/HAND™ apparatus described above, in accordance with a preferred embodiment. Some parts of the system 100 depicted in FIGS. 2A-C may be provided at a remote location. For example, in some embodiments, a facility may include a patient's home equipped with a mobile rehabilitation device (e.g., the InMotion portable rehabilitation apparatus described above).

In a preferred embodiment, the system 100 provides one or more authorized users 102 with access to device data 500, including patient information, device utilization (e.g., how much a device is used), patient evaluation assessment results, and therapy protocol exercise results. In an additional embodiment, the system 100 facilitates communication between the one or more authorized users 102 (e.g., a health care provider, a patient, the patient's family and/or friends) and the rehabilitation device 10 via a secure connection module 200.

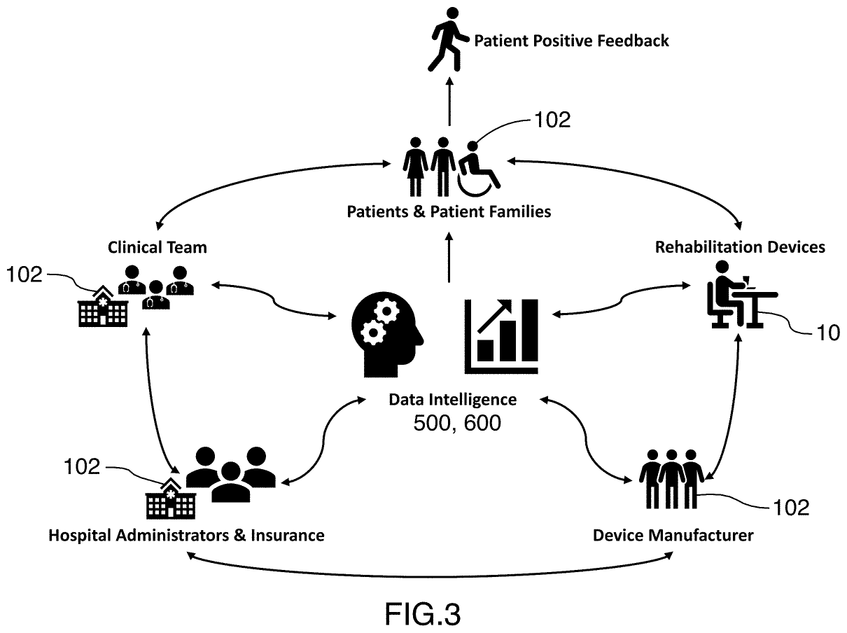

In a preferred embodiment, authorized users 102 include all stakeholders associated with the care of a patient as shown in FIG. 3, including, but not limited to: the patient; patient family and friends; clinical team; rehabilitation managers and/or directors; corporate management and/or administrators of the rehabilitation facility; insurance companies (e.g., claims administrators); Medicare coverage administrators; clinical support and training team for the device; and/or field service team for the device. Providing stakeholders with access to the device data 500 preferably generates a powerful feedback loop which operates to maximize patient outcomes and satisfaction on multiple levels. For example, providing the device data 500 and/or reports 600 to the clinical team (e.g., therapist) and management (e.g., therapy manager, director, CEO of a facility) associated with the successful use or adoption of the device 10 across clinicians and/or facilities (e.g., hospitals) preferably leads to the advantages of transparency and high visibility (e.g., drives compliance by staff) compared with the prior art which may be a manual (i.e., hands-on) and low technology approach. In addition, the provision of the device data 500 and/or reports 600 to the device provider preferably provides advantages related to tracking device utilization, patient outcomes and the ability to automatically provide support to enhance compliance, utilization and optimize outcomes. In a preferred embodiment, the type of device data 500, including patient information, that may be presented to various stakeholders is customizable.

In a preferred embodiment, designating patient family and friends as an authorized user 102 to access device data 500, for example, therapy accomplishments (e.g., rehabilitation progress, goal attainment, quick return to function, prognosis and knowledge of current status) will facilitate patient pride, excitement, and further encourage patient adoption of technology (e.g., increased use of the rehabilitation device and/or additional technology-based rehabilitation tools) to increase the probability of better patient rehabilitation outcomes.

In a preferred embodiment, designating the clinical team as an authorized user 102 to access device data 500, for example, real-time device usage metrics will facilitate increased oversight by a local rehabilitation manager and/or director on the usage amount of the devices within a given rehabilitation facility. If the usage amount is below a predetermined threshold value, the local rehabilitation manager and/or director could encourage the clinical team to increase use of the rehabilitation device amongst patients. Additional advantages include, for example, training and proficiency with the device, efficient evaluation and treatment of the patient, and ease of access to supporting device and/or rehabilitation documentation.

In a preferred embodiment, designating the corporate management team as an authorized user 102 to access device data 500, for example, real-time device usage metrics and/or patient information will facilitate increased oversight by the management team within a given rehabilitation facility to encourage the clinical team to increase use of the rehabilitation device amongst patients and continue to invest in the rehabilitation devices. Additional advantages include, for example, the availability of patient outcomes information and the payment of insurance claims.

In a preferred embodiment, designating the device manufacturer (or device provider) and/or system administrator as an authorized user 102 will facilitate the dissemination of expert clinical guidance (e.g., results from the analysis of data collected from all devices) and continuous development and improvement of the device (e.g., hardware and software).

Accordingly, the designation of the various stakeholders as authorized users 102, preferably generates one or more positive feedback loops with respect to patient rehabilitation outcomes. Preferably, the one or more feedback loops are adapted to optimize the utilization of technology, optimize the level of patient engagement with rehabilitation, promote alignment of rehabilitation across an organization, targets need to link patient centric rehabilitation results to patient management portals and electronic medical records at any hospital or rehabilitation facility.

In a preferred embodiment, the system 100 of the present invention is adapted to generate and display the device data 500, including utilization information (e.g., dashboards presenting device usage metrics), and reports 600, including therapy reports for one or more patients, evaluation reports for one or more patients, and/or patient accomplishment metrics (e.g., metrics and/or graphics depicting patient progress towards predetermined clinical goals) to enhance one or more feedback loops and further optimize device engagement.

In a preferred embodiment, the system 100 generates a technology-based therapeutic solution for the rehabilitation ecosystem.

In a preferred embodiment, the system 100 optimizes patient rehabilitation by providing the clinical team (e.g., practitioners) with device data 500, including patient information and treatment results for informed decision making, customized reporting capabilities in the platform focus on facility and organization measurement dashboards to support effective decision making for clinicians as well as hospital management. The dashboards are preferably adapted to present data 500 and/or reports 600 dynamically (i.e., in real-time, updatable and/or customizable).

System Architecture

In a preferred embodiment, referring to FIGS. 2A-C, there is shown a system 100 for use with a hospital organization 702 (which may include a number of hospitals from 1-n), an organization 704 (which may include multiple hospitals), an individual hospital 706, and other location(s) 708 (collectively, the “rehabilitation subsystem 700”); a physical or cloud server 802 within the administrator subsystem 800; and clients and stakeholders 102 (collectively, within the “accessing entity subsystem 900”). Some parts of the system 100 depicted in FIGS. 2A-C may be provided at a remote location.

In a preferred embodiment, the system 100 is used with one or more secure connection modules 200. Each of the one or more secure connection modules 200 may facilitate communication between one or more devices 10 and/or one or more servers 802 via satellite networks, terrestrial wireless networks, the Internet, and/or cloud computing platforms, among others. The communication of data (including, for example device data 500, updated configuration data 502) between the rehabilitation subsystem 700, the accessing entity subsystem 900 and the administrator subsystem 800 may also be achieved via one or more wired means of transmission or other physical means of transmission. Persons having ordinary skill in the art will appreciate the system 100 includes hardware and software.

Rehabilitation Subsystem

FIGS. 2A-B schematically illustrate, among other things that the rehabilitation subsystem 700 includes one or more devices 10 (alternately “robots 10”) associated with hospitals 706 (or other healthcare facilities). Each hospital 706 may be associated with a hospital organization 702, an organization 704 or be an individual hospital 706. Each of the one or more devices 10 is associated with a secure connection module 200 adapted to facilitate communication and/or transmission of data (e.g., device data 500) to the administrator subsystem 800. In some embodiments of the present invention, each secure connection module 200 may be associated with one or more devices 10.

The rehabilitation subsystem 700 (e.g., located at the hospitals, clinics, etc.) preferably generates device data 500 (as described in greater detail below). Each of the devices 10 generates device data 500 which preferably includes patient therapy session execution and therapy session information. The rehabilitation subsystem 700 may further include a device processor 24 (shown in FIG. 1) operative to execute the patient facing rehabilitation application 36, as well as gathering and transmitting device data 500 (including session information) to the administrator subsystem 800 (alternately “central repository 800”). In some preferred embodiments, the patient facing rehabilitation application 36 may be provided as a cross-platform desktop application (using, for example, Electron) and an open-source and cross-platform JavaScript runtime environment may also be provided (using, for example, Node.js).

The rehabilitation subsystem 700 is preferably configured to include a forward proxy. Persons skilled in the art will appreciate that the forward proxy is a proxy for the outside world, so that the behaviour of the device 10 is unchanged whether or not it is deployed. The forward proxy preferably insulates each device 10 from networking and security concerns related to external communications (i.e., proxying of all device interactions with external systems, operating systems and network interface).

Administrator Subsystem

FIG. 2C schematically illustrates, among other things that the administrator subsystem 800 provides a physical or cloud server 802 and repository for processing and consolidating, respectively, the device data 500. In particular, the administrator subsystem 800 includes a software application 804 for consolidating and processing the device data 500 received from the rehabilitation subsystem 700, including: organization management data (stored in an organization database 806a); hospital management data (stored in a hospital database 806b); device management data (stored in a device database 806c or, alternatively, a robot database 806c); user ID management data (stored in a user database 806d); and therapy data management data (stored in a therapy data database 806e). In a preferred embodiment, the software application 804 includes an organization management sub-application 804a, a hospital management sub-application 804b, a robot (device) management sub-application 804c, a user ID management sub-application 804d, and a therapy data management sub-application 804e. The sub-applications 804a,b,c,d,e are adapted to facilitate the processing of the various device data 500 received form the rehabilitation subsystem 700.

The server 802 components of the administrator subsystem 800 preferably include three runtimes within the software application 804. In one preferred embodiment, the first runtime is a single database server adapted to simplify the deployment architecture. Preferably, the database tiers for all services are hosted in a single database server instance (excluding clustering of the database for redundancy, etc.). To facilitate independence of the service implementations, each service preferably uses its own dedicated database schema. The schemas preferably do not depend on, or have any knowledge otherwise of, one another and are deployed on separate database servers. Persons having ordinary skill in the art may appreciate that PostgreSQL may be employed as a database.

In one preferred embodiment, the second runtime is a user ID management service. The Keycloak identity server may be packaged as a Docker image and used as a standalone container instance. Persons having ordinary skill in the art will appreciate that Keycloak is an open source software product to allow single sign-on with identity management and access management for applications and services and that other identity management and access management software products may be used with the present invention. Persons having ordinary skill in the art will appreciate that Docker is a set of platform as a service products that use OS-level visualization to deliver software in packages called containers and that other platform as a service products may be used with the present invention.

In one preferred embodiment, the third runtime is application modules. The application service tier preferably includes several independent modules that are combined in a single deployment runtime. Each component module preferably implements a Vert.x vertical. The runtime is a Vert.x process that preferably combines the module verticals and connects them to an internal router. Requests made to the external HTTP interface of the router are forwarded to the internal modules according to the request path. The runtime is deployed as a standalone Docker container, for example. The modules are preferably independent without any reliance on being co-deployed (i.e., they do not share state and only access one another via APIs). Persons having ordinary skill in the art will appreciate that the design facilitates the potential for a more robust architecture (e.g., independent deployment, scaling, and/or releasing of the modules) in the future, as the business needs of the system evolve while allowing for a simpler deployment configuration.

In a preferred embodiment, the user ID management service 804d (i.e., second runtime) provides a persistent store of application users and credentials, user sign-on, user role and organization assignments, user account provisioning and maintenance (including credentials reset, account activation/deactivation). Persons skilled in the art may appreciate that Keycloak and OpenID Connect can be used for identity and access management and authentication layer respectively. Persons skilled in the art will appreciate that OpenID Connect is a simple identity layer on top of the OAuth 2.0 protocol, which allows computing clients to verify the identity of an end-user based on the authentication performed by an authorization server, as well as to obtain basic profile information about the end-user in an interoperable and REST-like (i.e., Representational state transfer) manner and that alternate forms of identity verification may be used in accordance with the present invention.

In a preferred embodiment, therapy data management service 804e provides for the reception and storage of session data uploaded by the devices, processing and enriching of session data (e.g., calculation of utilization and outcomes metrics), queries for providing dashboard metrics data to the administrator dashboard application (e.g., as generated by the software application 804), and/or auditing of user level access to utilization and outcomes via queries. Persons skilled in the art may appreciate that Java and Vert.x can be used as a platform for the development of applications and application framework, respectively.

In a preferred embodiment, the organization management service 804a provides organization and site management, device management, and/or device authentication. Persons skilled in the art may appreciate that Java and Vert.x can be used as a platform for the development of applications and application framework, respectively.

In a preferred embodiment, the administrator processor 802 is also operative to execute a static web site for accessing the Dashboard and Administrator web applications. Persons skilled in the art may appreciate that Vert.x and web applications can be used as a platform for the development of applications for use with the system.

Accessing Entity Subsystem

FIG. 2C schematically illustrates, among other things, that the accessing entity subsystem 900 provides a client 902 (alternatively “stakeholder facing application 902”) including one or more web application(s) 904 and one or more mobile application(s) 906 for use by authorized users 102, including a patient and patient family/friends/caregiver, clinician, hospital management team, and/or device provider (not shown). In a preferred embodiment, the organization database 806a, hospital database 806b, device database 806c, user database 806d, and therapy data database 806e may be accessed by authorized users 102 (or “stakeholders 102” or “accessing entities 102”), including patient, patient's family, patient's friends, clinician, and/or hospital management, via the one or more web/mobile application(s) 904, 906. The ability to access the various databases 806a,b,c,d,e associated with the administrator subsystem 800 preferably enhances the feedback loop to encourage patient rehabilitation. In a preferred embodiment, the accessing entity subsystem 900 includes an accessing entity processor 908 (i.e., tablet, smartphone, laptop, desktop, smart television, etc.) for executing the client 902.

In some embodiments, a management dashboard web application (e.g., for hospital management) is provided and adapted for management views and reports for utilization metrics for groups, sites, and/or devices. Persons skilled in the art may appreciate that the web application can be generated using HTML 5, CSS, Javascript, React+Redux, and/or UIKit.

In some embodiments, an administration web application is provided and adapted for organization provisioning and management and/or device provisioning and management. Persons skilled in the art may appreciate that the web application can be generated using HTML 5, CSS, Javascript, React+Redux, and/or UIKit.

System Infrastructure

One-Way

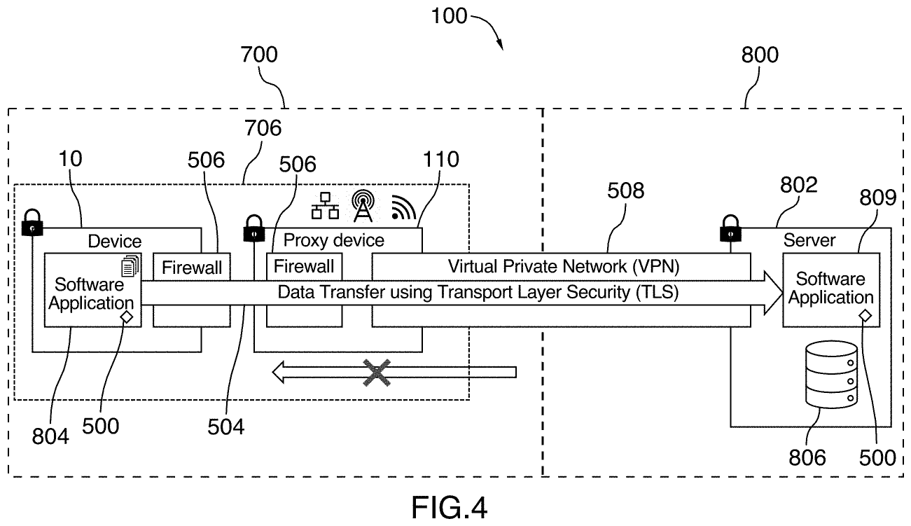

FIG. 4 depicts the infrastructure of the system 100 adapted for one way data flow 504 from one or more devices 10 to the server 802 that is HIPAA (Health Insurance Portability and Accountability Act) compliant and secure, in accordance with a preferred embodiment, wherein the rehabilitation subsystem 700 includes the device 10 and secure connection module 200 for one-way transmission of device data 500. The device 10 is preferably adapted to: (i) anonymize device data 500 (e.g., patient identifying information) for HIPAA compliance; (ii) provide secure automatic upload of device data 500 to the administrator subsystem 800 after each patient session; (iii) provide a firewall 506 to prevent data entry and securely connect to the administrative subsystem 800; and/or (iv) have the device 10 authenticate the server 802 prior to uploading any device data 500. In an alternate embodiment, at least some of the device data 500 (e.g., patient information) may not be anonymized prior to data transfer. The secure connection module 200 (or “proxy device 200”) is preferably adapted to provide: (i) a secure connection to the cloud server 802 using, for example, a cellular connection (e.g., speed up to 50 mbps); (ii) a Virtual Private Network (VPN) 508 to connect to the administrator subsystem 800 for additional security; and (iii) a Global SIM to facilitate communication of the devices 10 globally. In an alternate embodiment, the system 100 may not include a global SIM.

In a preferred embodiment, the system 100 is adapted to prevent backwards flow of data (i.e., one-way data flow only). In addition, the VPN 508 and firewall 506 prevent unauthorized access (i.e., access by a third party who is not an authorized user) to the one or more devices 10.

In a preferred embodiment, the system 100 is adapted to include a secured network using, for example, TLS encryption of data over VPN 508 and encryption using an AE256 algorithm. Persons having ordinary skill in the art will understand that other methods of encrypting data may be used in accordance with the present invention.

In a preferred embodiment, the administrator subsystem 800 is provided by a server 802 (e.g., a physical server, a cloud server, etc.). The system 100 preferably uses a cloud agnostic software application 804 in association with the device 10 and the server 802.

Two Way—Sending Update Data Back to Devices

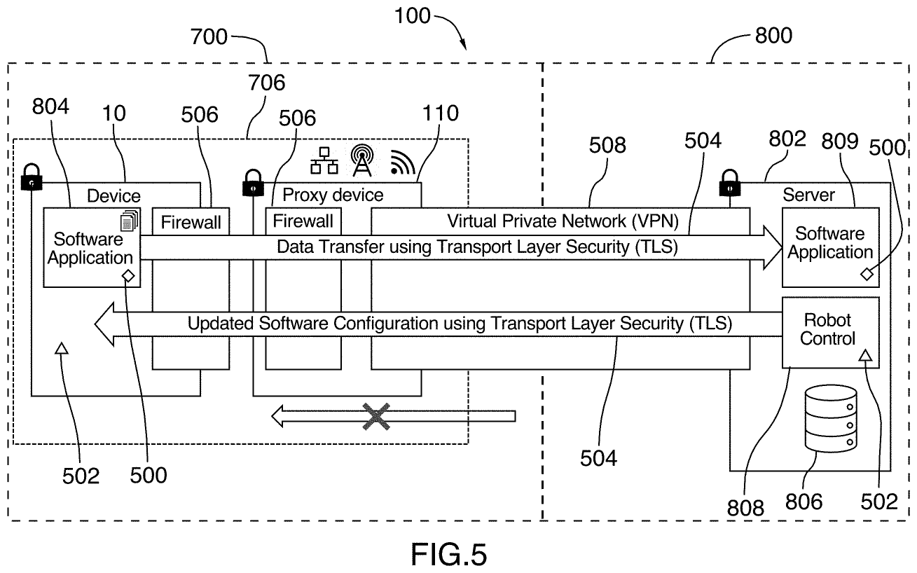

FIG. 5 depicts the infrastructure of the system 100 for bi-directional data flow 504 between the device 10 and the server 802 in accordance with a preferred embodiment, wherein the rehabilitation subsystem 700 (e.g., in a hospital 706) includes the device 10 and secure connection module 200 for two-way transmission of data (e.g., update data). The device 10 is preferably adapted to: (i) provide secured automatic device data 500 uploads to the administrator subsystem 800 after each patient session; (ii) receive update configuration 502 (alternatively, “update data 502”) from the administrator subsystem 800; (iii) provide a firewall 506 to prevent unauthorized data entry and securely connect to the administrator subsystem 800; and (iv) have the device 10 authenticate the server 802 prior to uploading any device data 500 containing identifying patient information. The secure connection module 200 preferably provides: (a) a secure connection to the administrator subsystem 800 (e.g., using WiFi or Cellular with speed up to 50 mbps); (ii) a Virtual Private Network 508 (or “VPN 508”) to connect to the administrator subsystem 800 for additional security; and (iii) provide a Global SIM to connect the devices globally. In an alternate embodiment, the system 100 may not include a global SIM.

In a preferred embodiment, the update data 502 (alternately, “Update Software Configuration 502”) is transmitted to the device 10 using a secure network.

In a preferred embodiment, communication between the rehabilitation subsystem 700 and the administrator subsystem 800 is accomplished using a secured network including, for example, TLS encryption of data over VPN 508 and encryption using an AE256 algorithm, to prevent unauthorized access. Persons having ordinary skill in the art will understand that other methods of encrypting data may be used in accordance with the present invention.

In a preferred embodiment, the administrator subsystem 800 includes a server 802 (e.g., a traditional physical server, a cloud server, etc.) adapted to receive device data 500 from the device. The server 802 preferably uses an agnostic software application 804. The server 802 preferably includes a robot control module 808 (or “device control module 808”) adapted to send updated configuration data 502 (e.g., updated configurations) to the device 10.

Dataflow from Inpatient Rehabilitation Facility to Outpatient Rehabilitation Facility

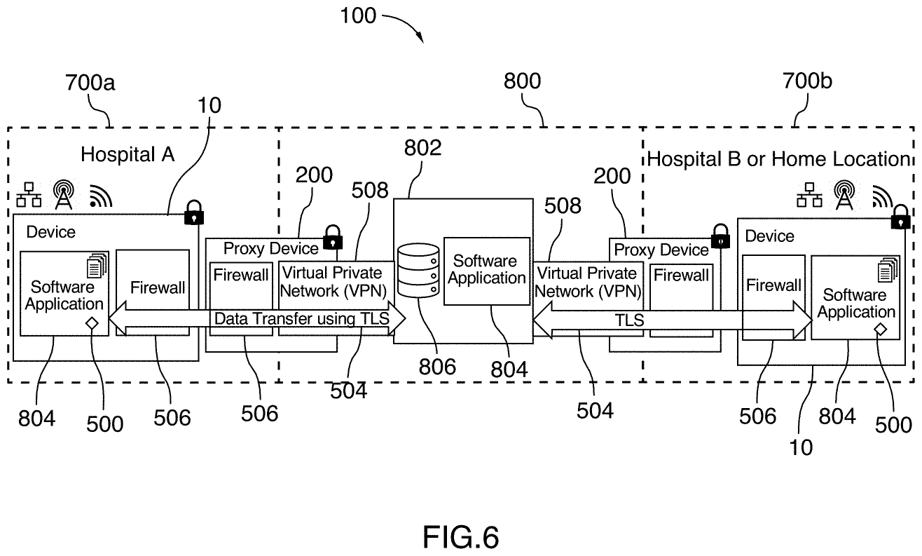

FIG. 6 depicts the infrastructure of the system 100 for data flow 504 between device 10 from location-to-location in accordance with a preferred embodiment, wherein a first rehabilitation subsystem 700a (e.g., one healthcare facility, such as Hospital A) includes the device 10 and secure connection module 200 for transmission of predetermined device data 500 (e.g., patient information) to a second rehabilitation subsystem 700b (e.g., another health care facility, such as Hospital B, or alternatively a Home Location) via the administrator subsystem 800 (e.g., as may occur when a patient is transferred between facilities or discharged to home). The rehabilitation subsystems 700a,b preferably include a patient database (not shown; residing at a local or remote location). The device data 500 generated by the device 10 in the first rehabilitation subsystem 700a is transmitted from the device 10 and/or patient database to a secure database 806 in the administrator subsystem 800 and then transferred to the patient database and/or device 10 in the second rehabilitation subsystem 700b.

In a preferred embodiment, communication between the first rehabilitation subsystem 700a, the administrator subsystem 800 and the second rehabilitation subsystem 700b are via a secured network with TLS encryption of device data over VPN and encryption using an AE256 algorithm. Persons having ordinary skill in the art will understand that other methods of encrypting data may be used in accordance with the present invention.

In a preferred embodiment, at least some device data 500 (e.g., patient identifying information) is anonymized during device data transfer 504 (e.g., secure HIPPA compliant data transfer) and a decryption key (not shown) is preferably associated with the anonymized patient record. The decryption key is applied by the second rehabilitation facility 700b following patient transfer between facilities. Accordingly, device data 500 (including, for example, patient identifying information and therapy data) is transferred from facility-to-facility to maintain the continuum of care.

In a preferred embodiment, the administrator subsystem 800 includes a server 802 (e.g., a traditional physical server, a cloud server, etc.). The server 802 (alternatively “administrator processor 802”) preferably uses an agnostic software application 804.

A person having ordinary skill in the art would understand that the data flow 504 depicted in FIG. 6, in addition to the continuum of care, can be maintained between the devices 100 associated with Hospital A, Hospital B and the patient's home.

Analysis of Device Data

In a preferred embodiment, the administrator subsystem 800 includes a processor 802 (i.e., the server 802) configured to analyze the data 500 using, among others, the report algorithm (alternatively, the “administrator algorithm”). The accessing entity 102 preferably has the ability to customize the analysis of the device data 500 based on factors, such as, patient diagnosis, patient ongoing performance, patient characteristics (e.g., sex, size, weight, etc.), patient personality profiles (e.g., social media, interests, etc.). The output of the device data analysis is preferably presented in one or more reports 600, including a management report 600a, a robot utilization report 600b, an outcome report 600c, an individual patient evaluation and therapy log report 600d, an individual patient therapy report 600e, and an individual patient evaluation report 600f.

In a preferred embodiment, the management report 600a is accompanied by the robot utilization report 600b, the outcome report 600c, and the patient evaluations and therapy log report 600d (directed towards clinicians). The robot utilization report 600b and the outcome report 600c preferably include all patients in a specific time range.

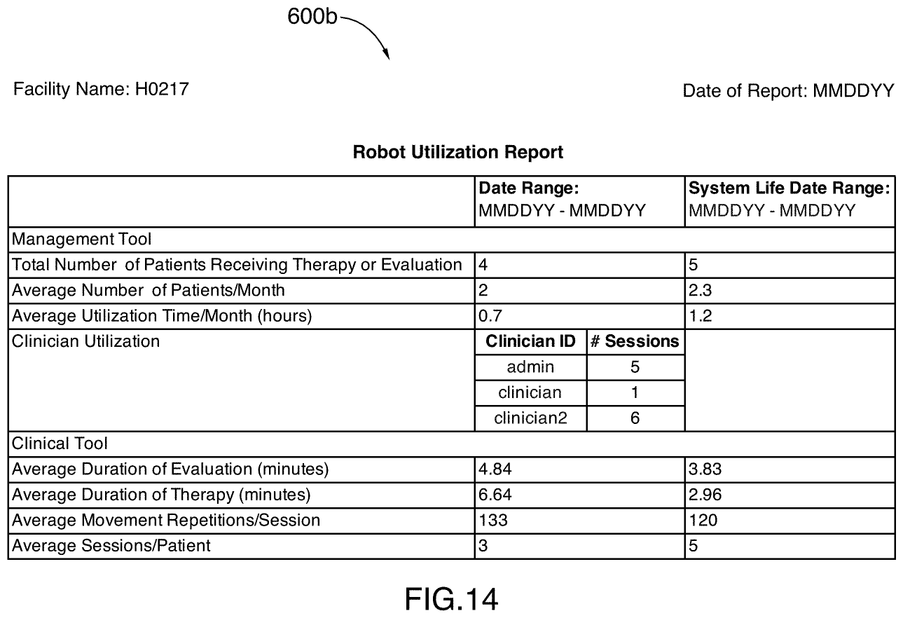

In a preferred embodiment, as shown in FIG. 14 based on mock data, the robot utilization report 600b includes a management tool and a clinical tool. The management tool may be customizable to any date range and include “life of robot metrics” such as patient number per month, robot session time per month, and clinician utilization (by clinician). The clinical tool may include duration of evaluation and therapy sessions, average movement repetitions per session, and average sessions per patient.

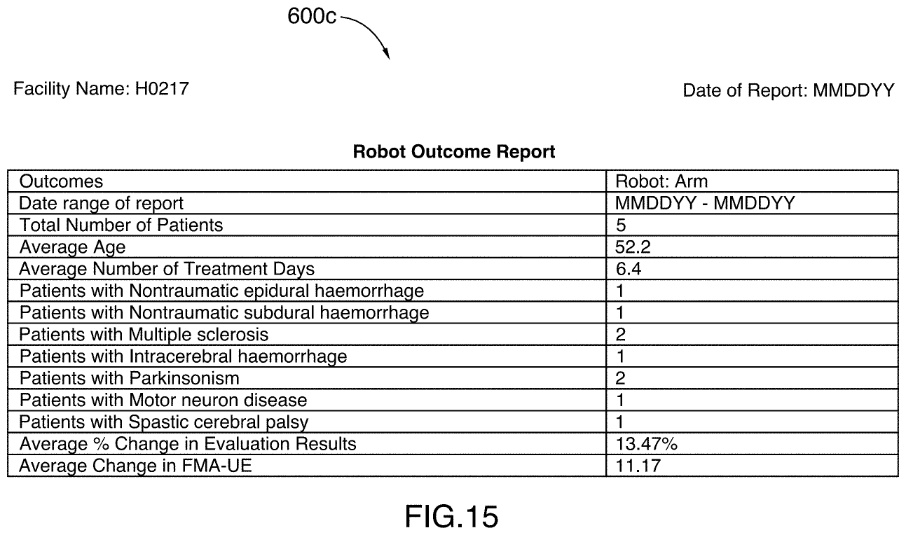

In a preferred embodiment, the outcome report 600c, as shown in FIG. 15 based on mock data, includes a configurable date range, patient information (e.g., total number of patients, average age, average number of treatment days), a percent change in evaluation results, and average change in FMA-UE (i.e., Fugl-Meyer Assessment Upper Extremity) results. Patients may include those with nontraumatic epidural haemorrhage, nontraumatic subdural haemorrhage, multiple sclerosis, intracerebral haemorrhage, parkinsonism, motor neuron disease, and/or spastic cerebral palsy.

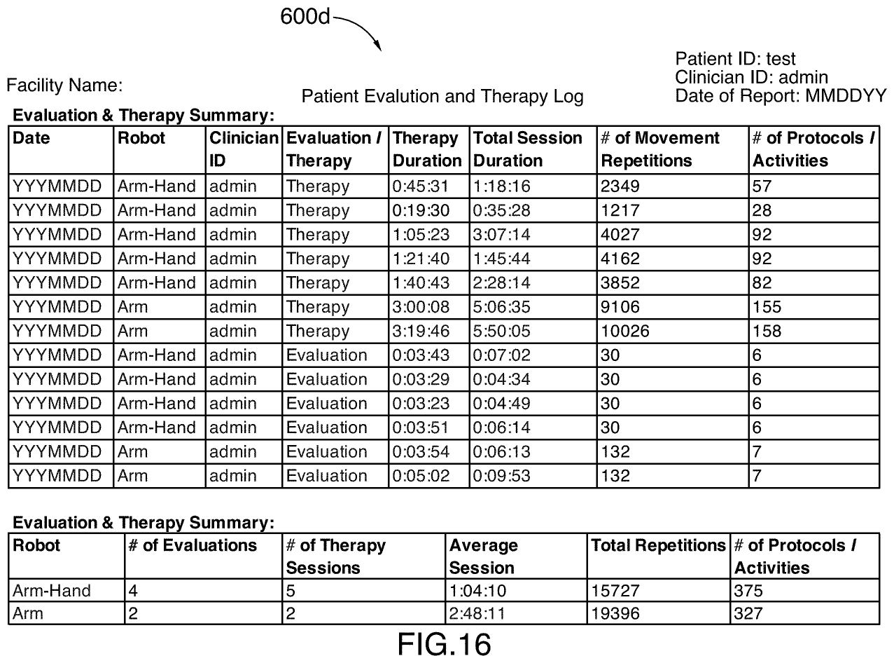

In a preferred embodiment, as shown in FIG. 16 based on mock data, the individual patient evaluation and therapy log report 600d includes the full period of individual patient experience with the device and covers all evaluation and therapy activities. The log preferably includes session dates, type of device, clinician ID, evaluation/therapy, therapy duration, total session duration, number of movement repetitions, and/or number of protocols/activities. The evaluation and therapy summary includes, device type, number of evaluations, number of therapy sessions, average session duration, total repetitions, number of protocols/activities.

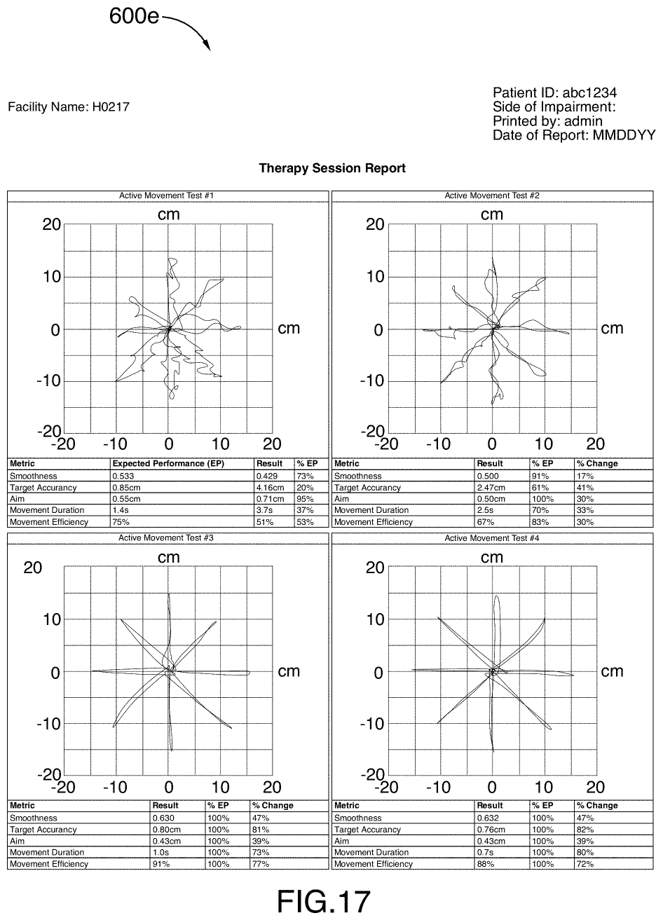

In a preferred embodiment, as shown in FIG. 17 based on mock data, the individual patient therapy report 600e is a single therapy session report for a device adapted to rehabilitate an arm. The therapy report includes movement metrics such as smoothness, target accuracy, aim, movement duration, and movement efficiency. Persons skilled in the art will appreciate the following:
Movement Record: An ideal plot would look like an eight-pointed asterisk (*);
Smoothness: A measure of smoothness of arm movement. A larger % EP indicates an increased ability to control changes in speed. Smoothness is important for tasks such as independent feeding and cooking (moving a spoon from bowl to mouth or drinking from a cup);
Target Accuracy: The proximity to the outer targets reached by arm movements at the end at each target task. A larger % EP indicates a greater range of sustained arm movement. This measure is relevant to functional tasks, such as reaching for a glass or bottle in a cupboard or reaching for a door handle;
Aim: The average deviation from the straight-line path during arm movement. A larger % EP indicates more controlled, purposeful and direct movement. Straight-line accuracy is important during tasks such as cooking on a stove top, reaching for a glass in a cupboard or mobilizing with a walker or wheelchair;
Movement Duration: The average time taken to move toward the outer targets. A larger % EP indicates greater functional speed of reaching movement;
Movement Efficiency: The ratio of the total distance of the patient's movement path with respect to the straight-line distance between the targets. A larger % EP indicates a greater ability to directly move between two points; and
% Change: The change in performance between the current Active Movement test and Active Movement Test #1.

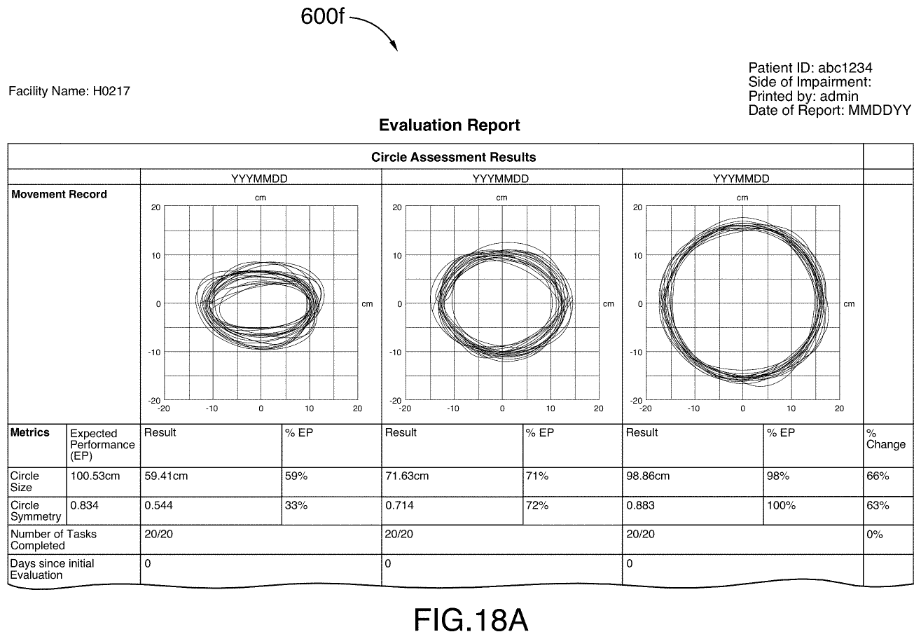

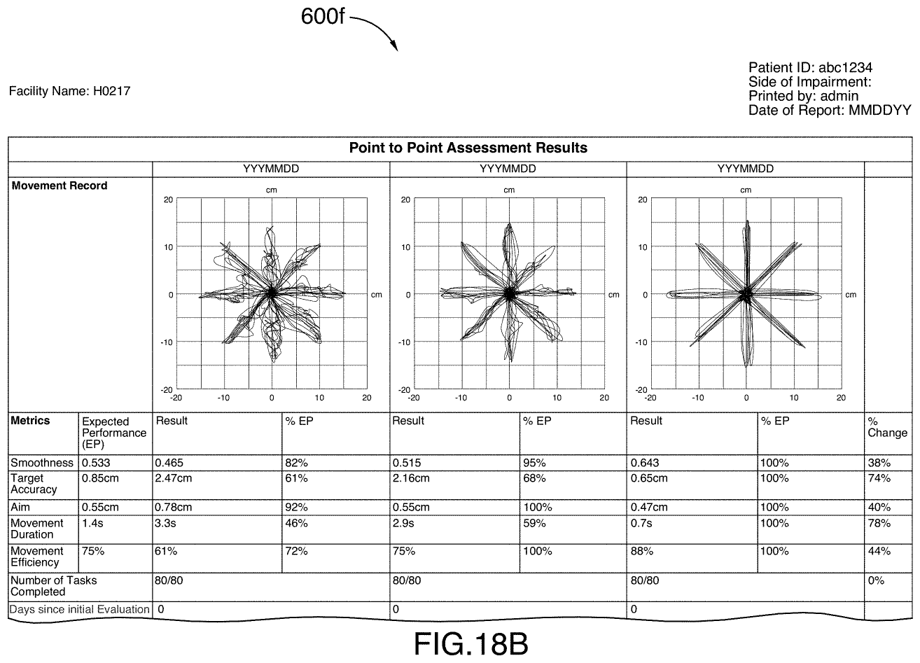

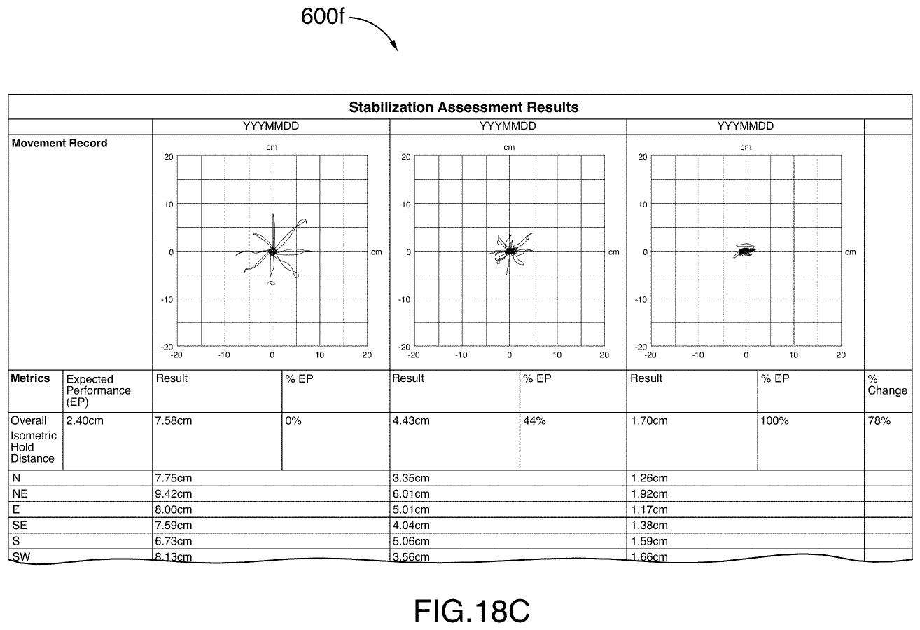

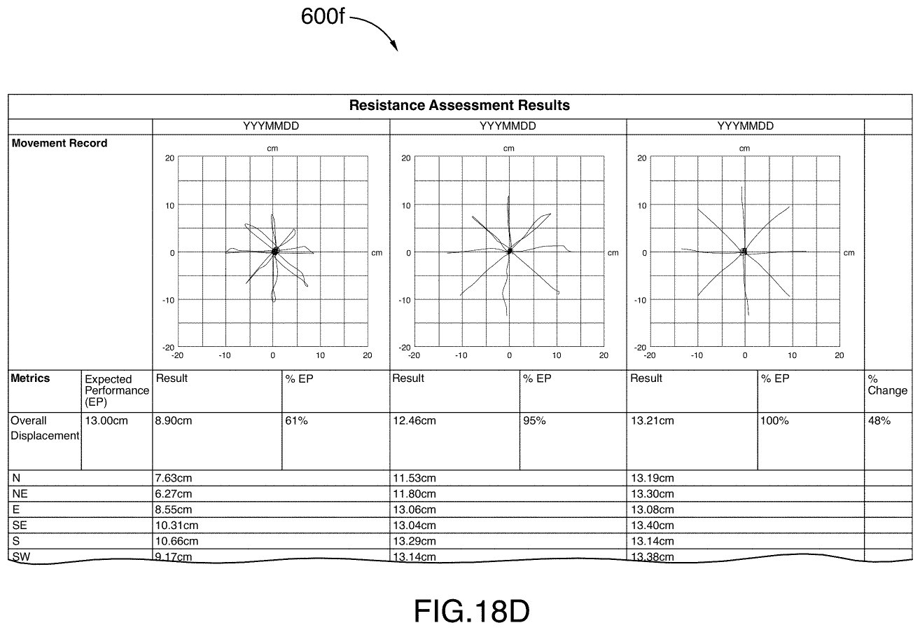

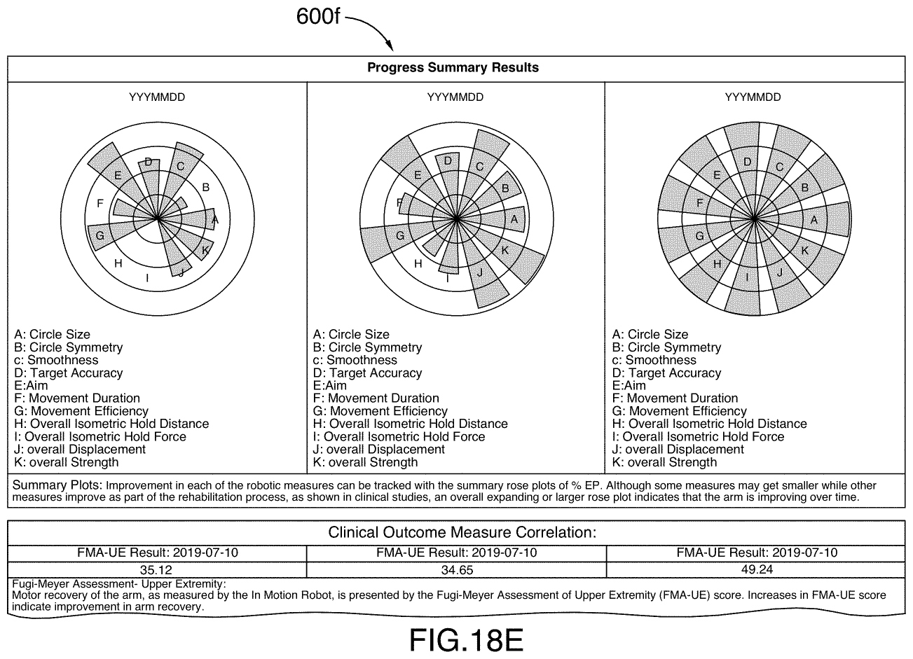

In a preferred embodiment, as shown in FIGS. 18A-E based on mock data, the individual patient evaluation report 600f is for a predetermined number of evaluations (e.g., three) and may include: a circle assessment (FIG. 18A); a point-to-point assessment (FIG. 18B); a stabilization assessment (FIG. 18C); a resistance assessment (FIG. 18D); and progress summary results (FIG. 18E).

Persons having ordinary skill in the art will appreciate the following metric summary for the circle assessment shown in FIG. 18A:
Circle Assessment: Tests the range of motion of the arm away from the body (out of the flexor synergy) and the ability to coordinate a single smooth movement in multiple directions. The motions evaluated represent functional tasks, such as independent feeding and cooking (stirring a pot of soup) or mobilization using a manual or electric wheelchair.
Movement Record: An ideal plot would be a circle one line thick. More consistent circles indicate improved control, and larger diameter circles show improved range of motion.
Circle Size: A measure of the average circle circumference. A larger % EP indicates a greater range of motion.
Circle symmetry: A measure of roundness using the ratio of the horizontal circle diameter to the vertical diameter. A larger % EP indicates better control of arm movement.
Number of Tasks Completed: A measure of endurance. An increase in the number of tasks completed indicates improved endurance.
% Change: The change in performance between the oldest and most recent selected dates.

Persons having ordinary skill in the art will appreciate the following metric summary for the point-to-point assessment show in FIG. 18B:
Point-to-Point Assessments: Quantifies the motor control of the patient's arm;
Movement Record: An ideal plot would look like an eight-pointed asterisk (*) with consistent point-to-point line movement;
Smoothness: A measure of smoothness of arm movement. A larger % EP indicates an increased ability to control changes in speed. Smoothness is important for tasks such as independent feeding and cooking (moving a spoon from bowl to mouth or drinking from a cup);
Target Accuracy: The proximity to the outer targets reached by arm movements at the end of each target task. A larger % EP indicates a greater range of sustained arm movement. This measure is relevant to functional tasks, such as reaching for a glass or bottle in a cupboard or reaching for a door handle;
Aim: The average deviation from the straight-line path during arm movement. A larger % EP indicates more controlled, purposeful and direct movement. Straight-line accuracy is important during tasks such as cooking on a stove top, reaching for a glass in a cupboard or mobilizing with a walker or wheelchair;
Movement Duration: The average time taken to move toward the outer targets. A larger % EP indicates greater functional speed of reaching movement;
Movement Efficiency: The ratio of the total distance of the patient's movement path with respect to the straight-line distance between the targets. A larger % EP indicates a greater ability to directly move between two points;
Number of Tasks Completed: A measure of endurance. An increase in the number of tasks completed indicates improved endurance; and
% Change: The change in performance between the oldest and most recent selected dates.

Persons having ordinary skill in the art will appreciate the following metric summary for the stabilization assessment shown in FIG. 18C:
Stabilization Assessment: Tests how well the arm can stabilize or hold its position against an applied force. The ability to stabilize the arm is important during tasks such as holding open a heavy door or leaning on your arm when getting in and out of bed. The direction of greatest displacement indicates which muscle groups provide the least stabilization of the arm and can help the clinician prescribe relevant therapy and exercises for arm function;
Movement Record: An ideal plot would be a single dot at the center paint of the chart;
Overall Isometric Hold Distance: The maximum distance the arm moved from the start position when robot force was applied, averaged over the different directions. A larger % EP indicates greater co-contraction of the shoulder and elbow muscles;
Overall Isometric Hold Force: The peak force applied by the arm to hold position when robot force was applied, averaged over the different directions. A larger % EP indicates greater isometric muscle strength;
Number of Tasks Completed: A measure of endurance. An increase in the number of tasks completed indicates improved endurance; and
% Change: The change in performance between the oldest and most recent selected dates.

The metric summary may be based on the following exemplary metrics:

| | Expected | | | | | | | |
|---|---|---|---|---|---|---|---|---|
| | Performance | | % | | % | | % | % |
| Metrics | (EP) | Result | EP | Result | EP | Result | EP | Change |
| Overall | 2.40 cm | 7.58 cm | 0% | 4.43 cm | 44% | 1.70 cm | 100% | 78% |
| Isometric | | | | | | | | |
| Hold | | | | | | | | |
| Distance | | | | | | | | |
| N | | 7.75 cm | | 3.35 cm | | 1.26 cm | | |
| NE | | 9.42 cm | | 6.01 cm | | 1.92 cm | | |
| E | | 8.00 cm | | 5.01 cm | | 2.17 cm | | |
| SE | | 7.59 cm | | 4.04 cm | | 1.38 cm | | |
| S | | 6.73 cm | | 5.06 cm | | 1.59 cm | | |
| SW | | 8.13 cm | | 3.56 cm | | 1.66 cm | | |
| W | | 7.50 cm | | 4.35 cm | | 1.35 cm | | |
| NW | | 5.49 cm | | 4.05 cm | | 2.28 cm | | |
| Overall | 26.0N | 14.1N | 0% | 21.6N | 56% | 26.6N | 100% | 90% |
| Isometric | | | | | | | | |
| Hold Force | | | | | | | | |

| | | | | |
|---|---|---|---|---|
| N | 12.5N | 24.0N | 27.5N | |
| NE | 9.9N | 17.5N | 27.2N | |
| E | 13.9N | 18.9N | 24.4N | |
| SE | 13.5N | 23.1N | 26.7N | |
| S | 15.2N | 23.4N | 27.6N | |
| SW | 12.1N | 21.1N | 25.4N | |
| W | 17.1N | 23.1N | 26.6N | |
| NW | 18.1N | 21.8N | 27.6N | |
| Number of Tasks | 16/16 | 16/16 | 16/16 | 0% |
| Completed | | | | |
| Days since Initial | 0 | 0 | 0 | |
| Evaluation | | | | |

Persons having ordinary skill in the art will appreciate the following for the resistance assessment shown in FIG. 18D:
Resistance Assessment: Measures arm strength as the ability to move against resistance. The ability to move against resistance is important during tasks such putting on a heavy coat, pushing open a door, or pushing a walking frame or wheelchair over uneven ground. The direction of least displacement indicates the muscle groups with the least strength and can help the clinician prescribe relevant therapy and exercises for arm function;
Movement Record: An ideal plot would look like an eight-pointed asterisk (*);
Overall Displacement: The greatest distance the arm moved toward the outer targets, averaged over the different directions. A larger % EP indicates greater range of motion against the robot force;
Overall Strength: The peak force the patient applied to reach toward the outer targets, averaged over the different directions. A larger % EP indicates greater muscle strength;
Number of Tasks Completed: A measure of endurance. An increase in the number of tasks completed indicates improved endurance; and
% Change: The change in performance between the oldest and most recent selected dates.

The metric summary may be based on the following exemplary metrics:

| | Expected | | | | | | | |
|---|---|---|---|---|---|---|---|---|
| | Performance | | % | | % | | % | % |
| Metrics | (EP) | Result | EP | Result | EP | Result | EP | Change |
| Overall | 13.00 cm | 8.90 cm | 61% | 12.46 cm | 95% | 13.21 cm | 100% | 48% |
| Displacement | | | | | | | | |
| N | | 7.63 cm | | 11.53 cm | | 13.19 cm | | |
| NE | | 6.27 cm | | 11.80 cm | | 13.30 cm | | |
| E | | 8.55 cm | | 13.06 cm | | 13.08 cm | | |
| SE | | 10.31 cm | | 13.04 cm | | 13.40 cm | | |
| S | | 10.66 cm | | 13.29 cm | | 13.14 cm | | |
| SW | | 9.17 cm | | 13.14 cm | | 13.38 cm | | |
| W | | 10.13 cm | | 13.10 cm | | 13.17 cm | | |
| NW | | 8.46 cm | | 10.77 cm | | 13.03 cm | | |
| Overall | 26.0N | 18.0N | 62% | 25.7N | 98% | 27.4N | 100% | 53% |
| Strength | | | | | | | | |

| | | | | |
|---|---|---|---|---|
| N | 15.4N | 23.2N | 27.7N | |
| NE | 12.7N | 24.0N | 27.1N | |
| E | 17.3N | 26.4N | 26.9N | |
| SE | 20.8N | 27.6N | 27.6N | |
| S | 21.4N | 27.5N | 27.6N | |
| SW | 18.6N | 28.2N | 27.8N | |
| W | 20.4N | 26.7N | 27.0N | |
| NW | 17.0N | 21.7N | 27.6N | |
| Number of Tasks | 16/16 | 16/16 | 16/16 | 0% |
| Completed | | | | |
| Days since Initial | 0 | 0 | 0 | |
| Evaluation | | | | |

Persons having ordinary skill in the art will appreciate the following for the progress summary results shown in FIG. 18E:
Summary Plots: Improvement in each of the robotic measures can be tracked with the summary rose plots of % EP. Although some measures may get smaller while other measures improve as part of the rehabilitation process, as shown in clinical studies, an overall expanding or larger rose plot indicates that the arm is improving over time.
Fugl-Meyer Assessment—Upper Extremity: Motor recovery of the arm, as measured by the rehabilitation robot, is presented by the Fugl-Meyer Assessment of Upper Extremity (FMA-UE) score. Increases in FMA-UE score indicate improvement in arm recovery. Research studies indicate that a 5.25-point change is required for a minimal clinically important difference in chronic patients following stroke with minimal to moderate upper extremity impairment. (Page S J et al. 2012) A 10-point increase in FMA-UE corresponds with a 1.5-point change in discharge Functional Independence Measure. (Shelton et al. 2001). In the report presented in FIG. 18E, the initial FMA-UE score is 35.12 and the most recent is 49.24. This is a 14.12-point change.

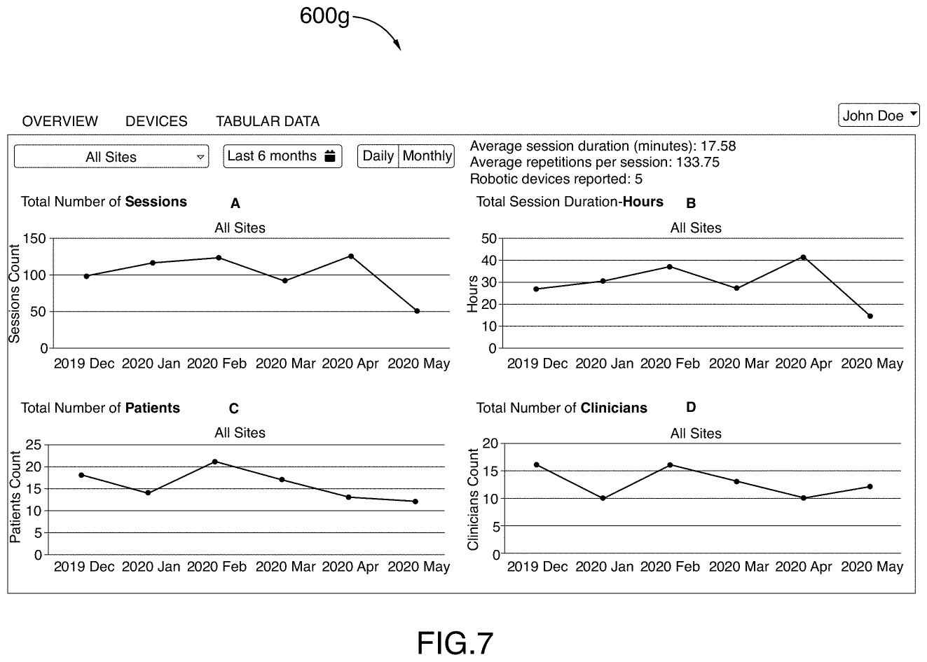

FIG. 7 is an example of an overview report 600g generated for all sites (i.e., hospitals) with monthly trend for a six (6) month period in accordance with a preferred embodiment. The report 600g includes an overview, information on specific devices, and tabular data options. The report 600g can be generated for a desired time-period (e.g., last 2 weeks, last 30 days, specific months, etc.) and may be toggled between monthly (FIG. 7) and daily. The report 600g preferably summarizes the average session duration (e.g., in minutes), the average repetitions per session, and/or the number of devices included in the report. Panel A displays the session count per month, Panel B displays the session duration (hours) per month, Panel C displays the patient count per month, and Panel D displays the clinicians count per month. The stakeholder for whom the report has been prepared is also displayed.

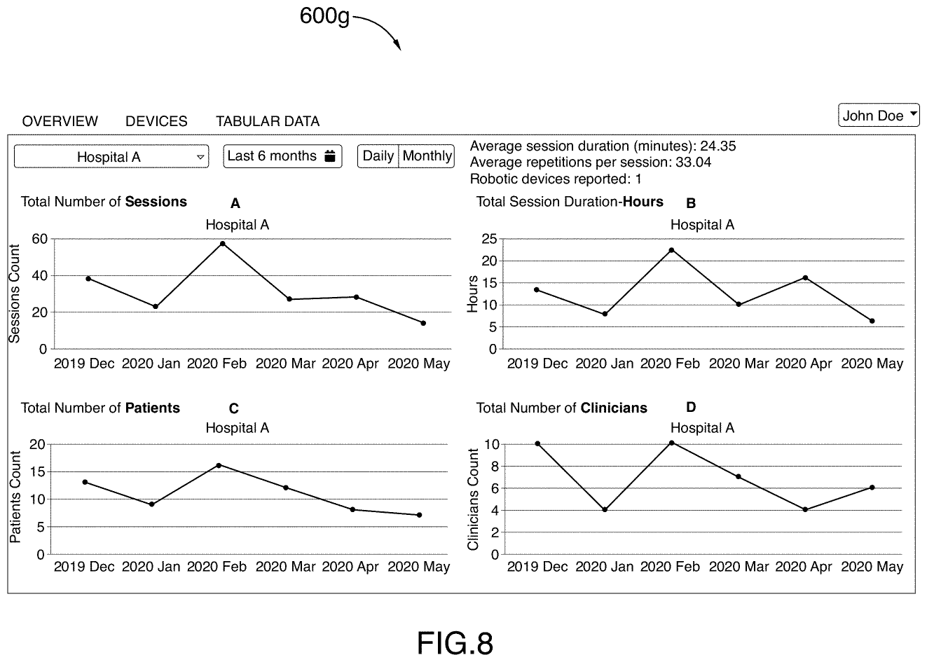

FIG. 8 is an example of an overview report 600g generated for a specific site (i.e., Hospital A) with monthly trend for a six (6) month period in accordance with a preferred embodiment. The report 600g includes an overview, information on specific devices, and tabular data options. The report 600g can be generated for a desired time-period (e.g., last 2 weeks, last 30 days, specific months, etc.) and may be toggled between monthly and daily. The report preferably summarizes the average session duration (e.g., in minutes), the average repetitions per session, and/or the number of devices included in the report. Panel A displays the session count per month, Panel B displays the session duration (hours) per month, Panel C displays the patient count per month, and Panel D displays the clinicians count per month. The stakeholder for whom the report has been prepared is also displayed.

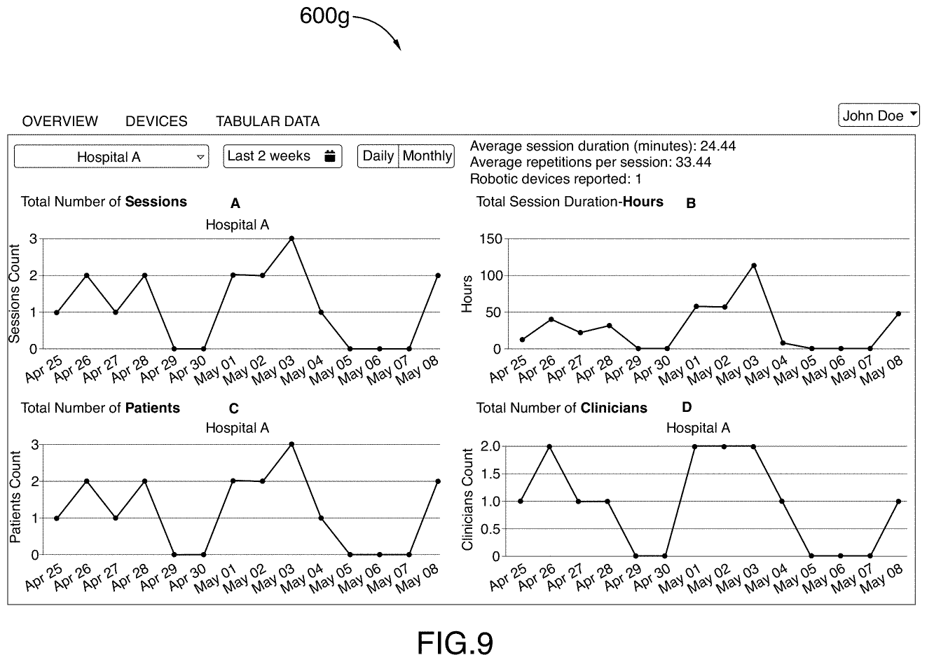

FIG. 9 is an example of the overview report 600g generated for a specific site (i.e., Hospital A) with daily trend for a two (2) week period in accordance with a preferred embodiment. The report 600g includes an overview, information on specific devices, and tabular data options. The report 600g can be generated for a desired time-period (e.g., last 2 weeks, last 30 days, specific months, etc.) and may be toggled between monthly and daily. The report 600g preferably summarizes the average session duration (e.g., in minutes), the average repetitions per session, and/or the number of devices included in the report. Panel A displays the session count, Panel B displays the session duration (minutes), Panel C displays the patient count, and Panel D displays the clinicians count. The stakeholder for whom the report has been prepared is also displayed.

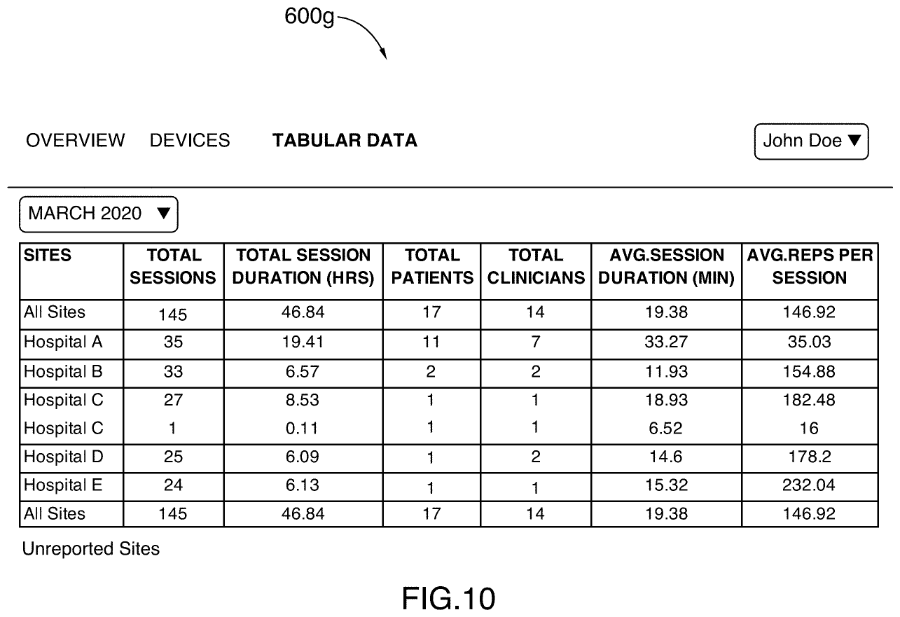

FIG. 10 depicts tabular data reporting in the overview report 600g for a given period of time (e.g., the month of October 2019) for multiple sites. The report 600g includes usage per site (e.g., total sessions, total usage (hours), total patients, total clinicians, average session duration (hours), and average repetitions per session). In addition, the report 600g lists sites that have not reported any device data.

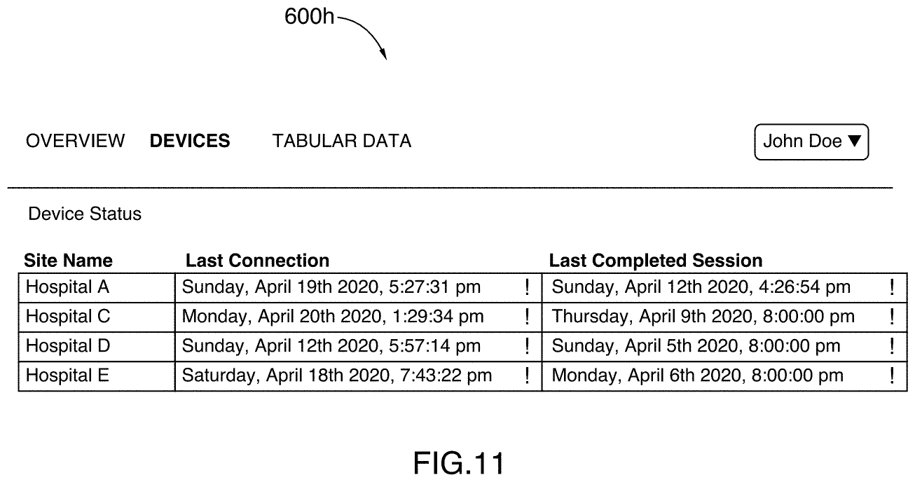

FIG. 11 is an example of a device status report 600h at each site, including the date and time of the last connection and the date and time of the last completed session (including flags or alerts when a device is not connected or not being used). In a preferred embodiment, selecting a site will re-direct the stakeholder to an overview page for the selected site.

In a preferred embodiment, the device data 500 and reports 600 may additionally be analyzed by the server 802 to identify patterns associated with patient outcomes depending on predetermined variables (e.g., comorbidities, age, gender, diagnosis, etc.) Such patterns are preferably applied by authorized users (or stakeholders) to one or more patients in order to further optimize patient outcomes with respect to rehabilitation (e.g., by generating additional rehabilitation protocols, identifying one or more rehabilitation protocols that will result in an optimal outcome, identifying one or more rehabilitation parameters and/or the appropriate values for the one or more parameters that will result in an optimal outcome).

Methods

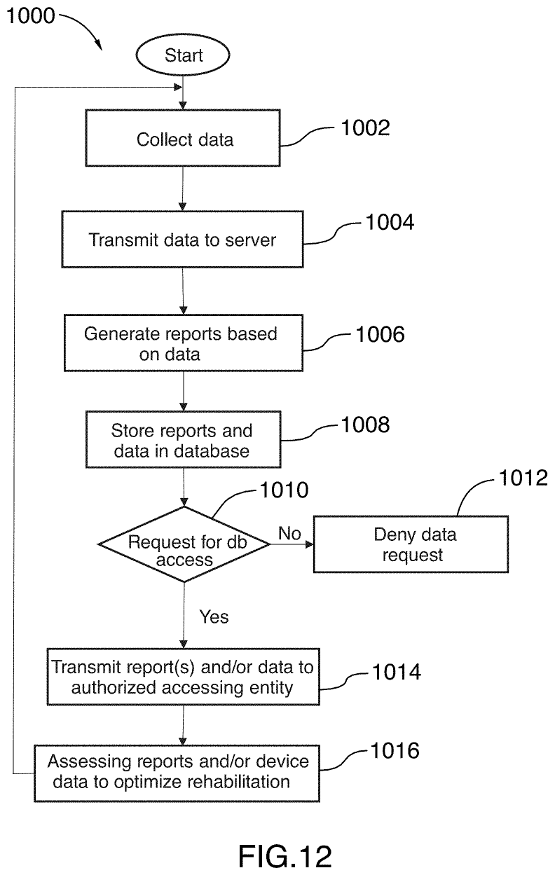

Referring now to FIG. 12, there is shown a flow diagram of an example method 1000 of optimizing patient rehabilitation in accordance with a preferred embodiment of the invention. The method 1000 may be carried out, for example, by the one or more processors 802 of the control system 800 (or the administrator subsystem 800 shown in FIG. 2). Preferably, the method 1000 is based on the patient's use of the device 10 in combination with the generation and analysis of device data 500 associated with the patient. The implementation of the method 1000 shown in FIG. 12 imparts a number of advantages for optimizing patient rehabilitation, including increased adoption of technology and visibility for the various authorized users 102, as compared to the prior art.

As shown in FIG. 12, the method 1000 includes the following steps, among others: a start step; a collect device data step 1002; a transmit device data to server step 1004; a generate reports based on analysis of data step 1006; a store device data and reports in administrator database step 1008; a request for access to reports and/or device data step 1010; a request denied step 1012 if the request is from an unauthorized accessing entity; a transmit reports and/or device data to the authorized accessing entity step 1014 if the request is from an authorized accessing entity. An assessing report and/or device data to optimize rehabilitation step 1016 (conducted, for example, by one or more stakeholders) concludes the method 1000. In preferred embodiments, as depicted in FIG. 12, the method 1000 may be repeated following the step of transmission of the reports and/or device data to the authorized accessing entity to promote a positive feedback loop on patient rehabilitation. The generate reports based on analysis of data step preferably generates the reports depicted in FIGS. 7-11, 14-17, and 18A-E.

In a preferred embodiment, each of the rehabilitation 700, the administrator 800 and the accessing entity 900 subsystems may be associated with one or more processors, one or more computer readable media (e.g., an onboard processor-readable memory, for example, a read-only memory (ROM) or dynamic random access memory (DRAM) which communicate with each other via a bus) local to the processor, one or more network interfaces (preferably including transmitter-receiver functions and adapted for use with a network), one or more databases, one or more input-output components, and one or more buses.

The processor may be a microcontroller, an embedded processor, a field programmable gate array (“FPGA”) or another suitable microprocessor. Preferably, the processor is operatively encoded with one or more algorithms stored in the memory, which provide the processor with, for example, one or more algorithms to provide logic to enable the processor to assess device data as well as any additional data that may be associated with the device and/or patient. In operation, processor receives data from the one or more devices, databases, input-output devices, and/or memory which may be on demand and/or at a predetermined time or time intervals (to, for example, drive real-time optimization of therapeutic sessions). The data is applied and/or implemented by the execution of certain algorithms (e.g., report generation algorithm) by the processor to generate output data (or reports), that includes predetermined metrics on device usage and patient rehabilitation progress.

The system includes various algorithm (e.g., report generation algorithm, encryption algorithm, decryption algorithm, patient anonymization algorithm, etc.), which causes the processor to perform any one or more of the instructions discussed herein. The system may include additional or different components, some of which may be optional and not necessary to provide aspects of the present disclosure. The system, as shown in FIGS. 2A-C, may be connected to other computing devices in a LAN, an intranet, an extranet, or the Internet. The system may operate in the capacity of a server or a client computing device in client-server network environment, or as a peer computing device in a peer-to-peer (or distributed) network environment. Further, while only a single processor is described, the term “processor” shall also be taken to include any collection of computing devices that individually or jointly execute a set (or multiple sets) of instructions to perform any one or more of the methodologies discussed herein.

System may further include a network interface device, one or more devices and one or more input-output devices (e.g., a keyboard and touch screen).

In an embodiment of the present invention, the system depicted in FIGS. 2A-C may be entirely or partially replicated remotely (e.g., cloud computing). Preferably, the remote system includes one or more remote processors capable of at least partially executing the method such that device data may be uploaded to the server and report data downloaded to the authorized accessing entity.

The computer readable medium stores executable instructions (i.e., algorithms) which, upon execution, analyzes device data, preferably received from the one or more devices, the input-output components and/or the database. The executable instructions provide logic to the processor for the performance of steps and/or to provide functionality as otherwise described above and elsewhere herein. The processor encoded by the computer readable medium are such as to perform an analysis on the device data to, for example, generate predetermined and/or desired reports. Thus, according to the invention, the computer readable medium facilitates the use of the processor to operatively facilitate the analysis of the device data. In alternate embodiments, the algorithm, may be transmitted or received over network via the network interface device.

Thus, the system, method, and computer readable medium operatively facilitate the optimization of patient rehabilitation.

The database includes, and is regularly updated with, the device data and reports. The database may be located behind a firewall relative to the network. Persons of ordinary skill in the art will appreciate that references herein to the database may include, as appropriate, references to: (i) a single database located local to the server; (ii) a single database located at a facility (e.g., remote to the server); and/or (iii) one or more congruent and/or distributed databases such as, for example, also including one or more sets of congruently inter-related databases—possibly distributed across multiple facilities.

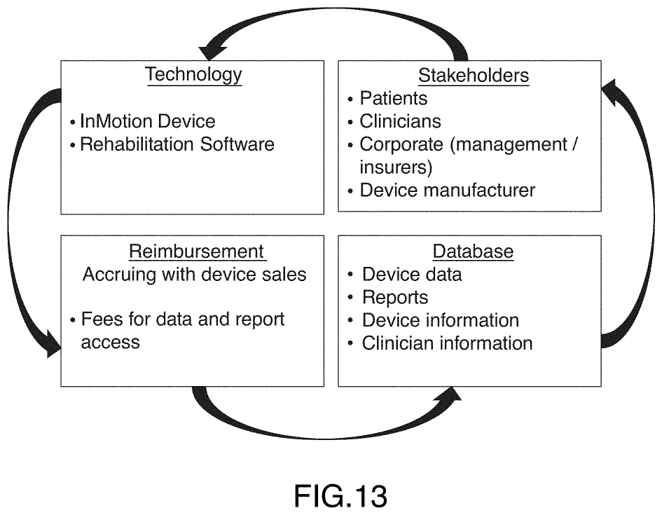

As shown by the diagram in FIG. 13, the system and method of device data collection is preferably used to create a self-sustaining and expanding business model based on circulating value. The model is based on four core assets: technology, reimbursement, database, and stakeholders.

The first core asset is the technology. The initial (inventive) technology may provide the initial drive for the business. The technology is preferably researched and developed to create the necessary hardware (e.g., a device such as an InMotion Robot) and software (e.g., proprietary rehabilitation software for use with the devices).

In particular, transmissions from the devices can provide clinical and/or other directly relevant information and can represent a new technology development Previously, the rehabilitation industry applied manual rehabilitation methods and patient progress was not readily visible to all stakeholders A device which bridges these two areas may be representative of the type of technology that drives the present business model.

The research and development of the technology leads to the creation of potential revenue streams through healthcare provider reimbursement and/or fees for data and report access generated by the adoption of the technology. A key aspect is that revenues may preferably scale with unit sales and use.

Next, data collection from the use of the technology may preferably be used to create one or more databases (whether a single congruent database, numerous mirror-image databases, and/or one or more distributed databases). The database(s) may preferably be linked to the growth of healthcare provider reimbursement—i.e., since reimbursement may preferably increase through the adoption of the technology, more information may be gathered and the resulting database may become larger and/or more powerful. The database may preferably, but need not necessarily, include device data, reports (as depicted in FIGS. 14, 15, 16, 17, and 18A-E), device information and clinician information.

As the database develops, the information may preferably be made available to numerous stakeholders (e.g., patients, clinicians, corporate (management/insurers), device manufacturer (service, clinical support), etc.), one or more of whom may then provide their viewpoints and/or an indication of their needs concerning the database Additional feedback may preferably be gained from users in the field (and/or potentially from the patients and/or clinicians) with respect to any hardware and/or software that may be currently deployed and/or used in conjunction with the system and method according to the present invention. One or more (and preferably all) aspects of the system and method may preferably tend toward a continual increase in the value of the database hardware and/or software of the database. Additionally, these aspects may also increase the value of the technology currently employed, and/or may help to set up a circulating knowledge management system, preferably to drive further developments in technology and optimization of patient rehabilitation.

The feedback gained may preferably be used to drive new technology research to address the issues raised. As expertise is developed in using the database, it may preferably become apparent that additional advancements and/or improvements to the technology might be required in order to gain more value from the database. Additional benefits may preferably include advantages gained from building alliances amongst stakeholders—e.g., (a) business alliances, (b) alliances as between similar or different businesses, industries, universities, hospitals, institutions, and/or governments, (c) social or support communities and alliances as between people having the same diseases, ailments or conditions. (d) community alliances, and/or (e) alliances within the same or similar communities. Furthermore, new and/or improved hardware and/or software might also preferably be developed, both to meet the needs of the users in the field and the requirements for the database, whether gleaned from or for use in the network communities and/or in communities of experts. The newly developed technologies may preferably help to fuel a new cycle, preferably including the development of: new healthcare reimbursement streams, improved databases, new and/or improved programming of the devices, and/or more stakeholder and patient feedback—any one or more of these factors driving still further new research and development. Preferably, the result is a circulating value business model which may provide for sustained reimbursement and growth from researched technologies.

The data collected from the devices, in use, may preferably be collated and organized into one or more databases as previously described herein. In addition to the data-mining capabilities provided by the database, it may also preferably provide a resource for expert stakeholders. These expert stakeholders, along with the users of the devices, may preferably generate feedback regarding the current technology, its use, and the content and efficiency of the one or more databases. This feedback may preferably help to direct the research into improvements in the devices, expansions (and/or refinements or improvements) in the scope of the current technology, and/or modifications to the database system and network.

The present disclosure may be described herein with reference to system architecture, block diagrams and flowchart illustrations of methods, and computer program products according to various aspects of the present disclosure. It may be understood that each functional block of the block diagrams and the flowchart illustrations, and combinations of functional blocks in the block diagrams and flowchart illustrations, respectively, can be implemented by computer program instructions.

These computer program instructions may be loaded onto a general purpose computer, special purpose computer, or other programmable data processing apparatus to produce a machine, such that the instructions that execute on the computer or other programmable data processing apparatus create means for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions specified in the flowchart block or blocks.

Accordingly, functional blocks of the block diagrams and flow diagram illustrations support combinations of means for performing the specified functions, combinations of steps for performing the specified functions, and program instruction means for performing the specified functions. It may also be understood that each functional block of the block diagrams and flowchart illustrations, and combinations of functional blocks in the block diagrams and flowchart illustrations, can be implemented by either special purpose hardware-based computer systems which perform the specified functions or steps, or suitable combinations of special purpose hardware and computer instructions.

In this disclosure, a number of terms and abbreviations may be used. The following definitions and descriptions of such terms and abbreviations are provided in greater detail.

It may be further generally understood by a person skilled in the relevant art that the term “downloading” refers to receiving datum or data to a local system from a remote system or to initiate such a datum or data transfer. Examples of a remote systems or clients from which a download might be performed include, but are not limited to, web servers, FTP servers, email servers, or other similar systems. A download can mean either any file that may be offered for downloading or that has been downloaded, or the process of receiving such a file. A person skilled in the relevant art may understand the inverse operation, namely sending of data from a local system to a remote system may be referred to as “uploading”. The data and/or information used according to the present invention may be updated constantly, hourly, daily, weekly, monthly, yearly, etc. depending on the type of data and/or the level of importance inherent in, and/or assigned to, each type of data. Some of the data may preferably be downloaded from the Internet, by satellite networks or other wired or wireless networks.

Elements of the present invention may be implemented with computer systems which are well known in the art. In general, computers include a central processor, system memory, and a system bus that couples various system components including the system memory to the central processor. A system bus may be any of several types of bus structures including a memory bus or memory controller, a peripheral bus, and a local bus using any of a variety of bus architectures. The structure of a system memory may be well known to those skilled in the art and may include a basic input/output system (“BIOS”) stored in a read only memory (“ROM”) and one or more program modules such as operating systems, application programs and program data stored in random access memory (“RAM”). Computers may also include a variety of interface units and drives for reading and writing data. A user of the system can interact with the computer using a variety of input devices, all of which are known to a person skilled in the relevant art.

One skilled in the relevant art would appreciate that the device connections mentioned herein are for illustration purposes only and that any number of possible configurations and selection of peripheral devices could be coupled to the computer system.

Computers can operate in a networked environment using logical connections to one or more remote computers or other devices, such as a server, a router, a network personal computer, a peer device or other common network node, a wireless telephone or wireless personal digital assistant. The computer of the present invention may include a network interface that couples the system bus to a local area network (“LAN”). Networking environments are commonplace in offices, enterprise-wide computer networks and home computer systems. A wide area network (“WAN”), such as the Internet, can also be accessed by a computer, a mobile device or the device.

It may be appreciated that the type of connections contemplated herein are exemplary and other ways of establishing a communications link between computers may be used in accordance with the present invention, including, for example, mobile devices and networks. The existence of any of various well-known protocols, such as TCP/IP, Frame Relay, Ethernet, FTP, HTTP and the like, may be presumed, and computer can be operated in a client-server configuration to permit a user to retrieve and send data to and from a web-based server. Furthermore, any of various conventional web browsers can be used to display and manipulate data in association with a web-based application. In addition, any of various mobile applications (including but not limited to iOS and Android applications) can be used to display and manipulate data.

The operation of the network ready device (i.e., a mobile device) may be controlled by a variety of different program modules, engines, etc. Examples of program modules are routines, algorithms, programs, objects, components, data structures, etc. that perform particular tasks or implement particular abstract data types. It may be understood that the present invention may also be practiced with other computer system configurations, including multiprocessor systems, microprocessor-based or programmable consumer electronics, network PCS, personal computers, minicomputers, mainframe computers, and the like. Furthermore, the invention may also be practiced in distributed computing environments where tasks are performed by remote processing devices that are linked through a communications network. In a distributed computing environment, program modules may be located in both local and remote memory storage devices.

Embodiments of the present invention can be implemented by a software program for processing data through a computer system. It may be understood by a person skilled in the relevant art that the computer system can be a personal computer, mobile device, notebook computer, server computer, mainframe, networked computer (e.g., router), workstation, processor onboard the device and the like. In one embodiment, the computer system includes a processor coupled to a bus and memory storage coupled to the bus. The memory storage can be volatile or non-volatile (i.e., transitory or non-transitory) and can include removable storage media. The computer can also include a display, provision for data input and output, etc. as may be understood by a person skilled in the relevant art.

Some portion of the detailed descriptions that follow are presented in terms of procedures, steps, logic block, processing, and other symbolic representations of operations on data bits that can be performed on computer memory. These descriptions and representations are the means used by those skilled in the data processing arts to most effectively convey the substance of their work to others skilled in the art. A procedure, computer executed step, logic block, process, etc. is here, and generally, conceived to be a self-consistent sequence of operations or instructions leading to a desired result. The operations are those requiring physical manipulations of physical quantities. Usually, though not necessarily, these quantities take the form of electrical or magnetic signals capable of being stored, transferred, combined, compared, and otherwise manipulated in a computer system. It has proven convenient at times, principally for reasons of common usage, to refer to these signals as bits, values, elements, symbols, characters, terms, numbers or the like.

It should be borne in mind, however, that all of these and similar terms are to be associated with the appropriate physical quantities and are merely convenient labels applied to these quantities. Unless specifically stated otherwise as apparent from the following description, it is appreciated that throughout the present invention, references utilizing terms such as “receiving”, “creating”, “providing”, “communicating” or the like refer to the actions and processes of a computer system, or similar electronic computing device, including an embedded system, that manipulates and transfers data represented as physical (electronic) quantities within the computer system's registers and memories into other data similarly represented as physical quantities within the computer system memories or registers or other such information storage, transmission or display devices.

The present invention is contemplated for use in association with one or more cooperating environments, to afford increased functionality and/or advantageous utilities in association with same. The invention, however, is not so limited.

Naturally, in view of the teachings and disclosures herein, persons having ordinary skill in the art may appreciate that alternate designs and/or embodiments of the invention may be possible (e.g., with substitution of one or more steps, algorithms, processes, features, structures, parts, components, modules, utilities, etc. for others, with alternate relations and/or configurations of steps, algorithms, processes, features, structures, parts, components, modules, utilities, etc.).

Although some of the steps, algorithms, processes, features, structures, parts, components, modules, utilities, relations, configurations, etc. according to the invention are not specifically referenced in association with one another, they may be used, and/or adapted for use, in association therewith.

One or more of the disclosed steps, algorithms, processes, features, structures, parts, components, modules, utilities, relations, configurations, and the like may be implemented in and/or by the invention, on their own, and/or without reference, regard or likewise implementation of one or more of the other disclosed steps, algorithms, processes, features, structures, parts, components, modules, utilities, relations, configurations, and the like, in various permutations and combinations, as may be readily apparent to those skilled in the art, without departing from the pith, marrow, and spirit of the disclosed invention.

While computer-readable storage medium may be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

It may generally be understood by a person skilled in the relevant art that the term “cloud computing” is an information technology model that facilitates ubiquitous access to shared pools of configurable system resources and higher-level services that can be provisioned with minimal management effort, usually over the Internet. Third-party clouds preferably enable organizations to focus on their core businesses instead of allocating resources on computer infrastructure and maintenance.

The methods, components, and features described herein may be implemented by discrete hardware components or may be integrated in the functionality of other hardware components such as ASICS, FPGAs, DSPs or similar devices. In addition, the methods, components, and features may be implemented by firmware modules or functional circuitry within hardware devices. Further, the methods, components, and features may be implemented in any combination of hardware devices and software components, or only in software.

In the present description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

The present disclosure also relates to an apparatus for performing the operations herein. This apparatus may be specially constructed for the required purposes, or it may include a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (“ROMs”), random access memories (“RAMs”), EPROMs, EEPROMs, magnetic or optical cards, or any type of media suitable for storing electronic instructions.

The foregoing description has been presented for the purpose of illustration and may be not intended to be exhaustive or to limit the invention to the precise form disclosed. Other modifications, variations and alterations are possible in light of the above teaching and may be apparent to those skilled in the art, and may be used in the design and manufacture of other embodiments according to the present invention without departing from the spirit and scope of the invention. It may be intended the scope of the invention be limited not by this description but only by the claims forming a part of this application and/or any patent issuing therefrom.

The embodiments for which an exclusive privilege or property is claimed are as follows:

1. A system for optimizing one or more clinical outcomes for a rehabilitation patient by a stakeholder, wherein the system comprises:
(a) a rehabilitation device for generating device data associated with the rehabilitation patient, the device comprising one or more rehabilitation processors operative to electronically receive and/or transmit the device data;
(b) one or more administrator processors operative to: (i) electronically receive the device data from the one or more rehabilitation processors; and (ii) automatically apply an administrator algorithm to the device data to generate one or more reports associated with the rehabilitation patient;
(c) one or more databases for electronically storing the device data and the one or more reports associated with the rehabilitation patient; and
(d) an accessing entity processor operative to execute a stakeholder facing application for reviewing at least a portion of the device data and/or the one or more reports associated with the rehabilitation patient from the one or more databases by the stakeholder;
whereby the system is operative to facilitate the optimization of the one or more clinical outcomes for the patient based on the review of the at least a portion of the device data and/or the one or more reports associated with the rehabilitation patient by the stakeholder.

2. The system of claim 1, further comprising a secure connection module to facilitate the secure communication of data between the one or more rehabilitation processors and the one or more administrator processors.

3. The system of claim 1, further comprising a plurality of rehabilitation devices.

4. The system of claim 1, further comprising two or more rehabilitation facilities.

5. The system of claim 1, further comprising update data and the one or more administrator processors are operative to transmit the update data to the one or more rehabilitation processors.

6. The system of claim 1, wherein the administrator processor is further adapted to transmit the device data received from the rehabilitation processor to a second rehabilitation processor.

7. The system of claim 1, wherein the reports include a management report, a robot utilization report, an outcome report, an individual patient evaluation and therapy log report, an individual patient therapy report, and/or an individual patient evaluation report.

8. The system of claim 1, wherein the rehabilitation patient includes patients with nontraumatic epidural haemorrhage, nontraumatic subdural haemorrhage, multiple sclerosis, intracerebral haemorrhage, parkinsonism, motor neuron disease, and/or spastic cerebral palsy.

9. The system of claim 1, wherein the stakeholder includes a patient, a patient family member, a patient friend, a clinician, a rehabilitation manager, a hospital manager, an insurer and/or a device manufacturer.

10. The system of claim 1, wherein the optimization of the one or more clinical outcomes for the patient includes an increase in a Rose plot and/or an increase in FMA-UE score associated with the rehabilitation patient.

11. A method for optimizing one or more clinical outcomes for a rehabilitation patient by a stakeholder, wherein the method comprises the steps of:
(a) operating a rehabilitation device to generate device data associated with the rehabilitation patient;
(b) operating one or more rehabilitation processors to collect the device data associated with the rehabilitation patient and electronically transmit the data;
(c) operating one or more administrator processors to electronically receive the device data from the one or more rehabilitation processors to: (i) automatically apply an administrator algorithm to the device data to generate one or more reports associated with the rehabilitation patient;
(d) electronically storing the device data and the one or more reports associated with the rehabilitation patient; and
(e) operating an accessing entity processor to execute a stakeholder facing application for reviewing at least a portion of the device data and/or the one or more reports associated with the rehabilitation patient from the one or more databases by the stakeholder;
wherein one or more clinical outcomes for the patient are optimized based on the review of the at least a portion of the device data and/or the one or more reports associated with the rehabilitation patient by the stakeholder.

12. The method of claim 11, further comprising a step of operating a secure connection module to facilitate the secure communication of data between the one or more rehabilitation processors and the one or more administrator processors.

13. The method of claim 11, further comprising a step of transmitting update data from the one or more administrator processors to the one or more rehabilitation processors.

14. The method of claim 11, further comprising a step of transmitting the device data received form the rehabilitation processor to a second rehabilitation processor.

15. The method of claim 11, wherein the reports include a management report, a robot utilization report, an outcome report, an individual patient evaluation and therapy log report, an individual patient therapy report, and/or an individual patient evaluation report.

16. The method of claim 11, wherein the rehabilitation patient includes patients with nontraumatic epidural haemorrhage, nontraumatic subdural haemorrhage, multiple sclerosis, intracerebral haemorrhage, parkinsonism, motor neuron disease, and/or spastic cerebral palsy.

17. The method of claim 11, wherein the stakeholder includes a patient, a patient family member, a patient friend, a clinician, a rehabilitation manager, a hospital manager, an insurer and/or a device manufacturer.

18. The method of claim 11, wherein the optimization of the one or more clinical outcomes for the patient includes an increase in a Rose plot and/or an increase in FMA-UE score associated with the rehabilitation patient.

19. A non-transient computer readable medium on which is physically stored executable instructions for use with optimizing one or more clinical outcomes for a rehabilitation patient by a stakeholder, wherein the executable instructions comprise processor instructions for one or more rehabilitation processors, one or more administrator processors and/or an accessing entity processor to automatically:
(a) generate device data associated with the rehabilitation patient;
(b) apply an administrator algorithm to the device data to generate one or more reports associated with the rehabilitation patient;
(c) electronically store the device data and the one or more reports associated with the rehabilitation patient in one or more databases; and
(d) execute a stakeholder facing application for reviewing at least a portion of the device data and/or the one or more reports associated with the rehabilitation patient from the one or more databases by the stakeholder;
wherein the review of the at least a portion of the device data and/or the one or more reports associated with the rehabilitation patient by the stakeholder are for use in optimizing the one or more clinical outcomes for the patient.
